SEQUENCE LISTING (13 sequences)

SEQ ID NO: 1 - DNA, 23 bp, synthetic construct
  source 1..23: mol_type = other DNA; note = Ptgds-sgRNA1
ccacgttgctggcctcaggctca

SEQ ID NO: 2 - DNA, 23 bp, synthetic construct
  source 1..23: mol_type = other DNA; note = Reverse complement of Ptgds-sgRNA1
tgagcctgaggccagcaacgtgg

SEQ ID NO: 3 - DNA, 23 bp, synthetic construct
  source 1..23: mol_type = other DNA; note = Ptgds-sgRNA2
ccaaagcaagtcagacctcggct

SEQ ID NO: 4 - DNA, 23 bp, synthetic construct
  source 1..23: mol_type = other DNA; note = Reverse complement of Ptgds-sgRNA2
agccgaggtctgacttgctttgg

SEQ ID NO: 5 - DNA, 15 bp, synthetic construct
  source 1..15: mol_type = other DNA; note = Nucleotide sequence of the intron sequence fragment
tgcccacgttgctgg

SEQ ID NO: 6 - DNA, 2935 bp, synthetic construct
  source 1..2935: mol_type = other DNA; note = Nucleotide sequence of the exon sequence fragment
cctcaggctcagacacctgctctactccaagcaaatggctgctcttccaatgctgtggaccgggctggtcctcttgggtctcttgggatttccacagaccccagcccagggccatgacacagtgcagcccaactttcaacaagacaaggtgagagggtcccctaccccacacccgaggaaacagaaacctcaggtcagagccaggctttctctcacaagagagggtgcgttgggcgctgtcagccatgggagctgtctggaaccgcgctggcacacagcctggttggtccacctgactccgccaggaatgtggctctgatacccactttaccggaagagtagactggggcgagcactgggacaaagacgggagctcaacatcctggggaaggaaggggtcaatgaggcaatgagccagcctactagagagagagaggggcgtggatgctaccagaacctgtgtgtgggaggagtcagagtagggaaggccagcccactagggtctgcccatgaggggcgcatggtgcagacccgggcatccactggtcacagttcctggggcgctggtacagcgcgggcctcgcctccaattcaagctggttccgggagaagaaagagctactgtttatgtgccagacagtggtagctccctccacagaaggcggcctcaacctcacctctaccttcctaaggtgagacaagggggtgtggcaagtttcgggacagaaggccccacaaccctgtctgggggacatcctggggcttgttcccttacatcaggggtaatctacccacaggaaaaaccagtgtgagaccaaggtgatggtactgcagccggcaggggttcccggacagtacacctacaacagcccccgtgagtgagccacttccttatctgggtaaattctgaggtaaatgctggcagactgtgcagccccctgtcccaaaaggtggggataatggtcacaccacaagggtcagtcatccaagaccagacctgattgtgaatctgcctcaggcacacagggctacctctctccagggactttggcctctctgaaacccagccacattcttccaggcccctttcctgtccaaatgaaatttcccagtactctgctgcccaagtgggtcacatacaggcattccccaaatcctacccacatttcatagctcctatccaagtacctctttccatgcctcacctgatctatggattcccaccagaaccctatttccttggccttcctgctatattgtaactcagcctgatgatttcttgagtctaagtgttttctgccctctccccaagattcatggtttggagttagtgttcaggaaggaagctagagattgggtggtggccacccaggggagcacagggaaagaagccaaagcaggggtggaggaggaaggcctgagaccctccccacagagaagcccacaaaggccaccccctccaagcagagggagatagtgatgtgggagccacatgtcttaatcagtgtcatttcttgggttcccagactggggcagcttccactccctctcagtggtagaaaccgactacgatgagtacgcgttcctgttcagcaagggcaccaagggcccaggccaggacttccgcatggccaccctctacagtaggtatcccagcccacaggcccacgcacagggcagatgcctgaggttggaaacagaccaaggcctaacccagaggacagtaacgaaggtgtgtgggggcagggcgagggcttttcacctcctgacaccggccccttctttatctaccaggcagagcccagcttctgaaggaggaactgaaggagaaattcatcacctttagcaaggaccagggcctcacagaggaggacattgttttcctgccccaaccgggtgagggaggctaagctgctgaggagggaattagtgcagattagtgcagcctgtggactggggagagtgtggccgcctactagtccaggggctccaaggaaagaaatggaggtgtcagtctgtcccgacagtacctcgacctgcagccccctttattgggaaccctcttcctggtggacacctcgctgccctgtctgccagccccctagctagggatttaggggcactaacagatggagaaagacaccttttatgttttaaagaacagattggagcaggagtgggatggagtctgaagtgtggggctcagccttggggaggcttcgtaaagtccagggagaagacaaagtcctggtgactgtgggtctaagcctgatactgactacttccctgggcttctttctcaacagataagtgcattcaagagtaaacacaggtgagagaagtcagtcacaggtaacacatggtaagtgccatttactcactcaacataagaccactgagtgctcatgtgaccacggagtgcgggctggggtgggggggatgcagctgcccaaggactgtccaagtgagacagccagagagaaaggacagttccaattccagtggcaggaatagagctgatggccaagggttcatgggagaaggataacagcaatgggaagggaccgccccatgaagcccatcctgcaaaatgagtctccaaggaaccagaatggacaagatcgggaagggactggtggccagggatggacatggcgagtcagagggctggctcctcacctgtgctgttgactgagactctgagaccataggccctggagggataccctaggaggccctggccggaagtgttgtttgggccccactgggctcagggtgctgccctcatcactgatggctcttgttcttctgtgcaggtgatgtggcctcaggactcccgtgctctgtcactcttgagacccaagccctggctccccaaagaccttctccgccctccagctttgccttggtggagaaataaaatccaaagcaagtc

SEQ ID NO: 7 - DNA, 20 bp, synthetic construct
  source 1..20: mol_type = other DNA; note = Primer Ptgds-L-S
gcaggtcttagccataggtg

SEQ ID NO: 8 - DNA, 18 bp, synthetic construct
  source 1..18: mol_type = other DNA; note = Primer Ptgds-L-A
tttctgtttcctcgggtg

SEQ ID NO: 9 - DNA, 21 bp, synthetic construct
  source 1..21: mol_type = other DNA; note = Primer Ptgds-R-S
gatggctcttgttcttctgtg

SEQ ID NO: 10 - DNA, 22 bp, synthetic construct
  source 1..22: mol_type = other DNA; note = Primer Ptgds-R-A
ccttccaactttattgcctaat

SEQ ID NO: 11 - DNA, 4299 bp, synthetic construct
  source 1..4299: mol_type = other DNA
tagcctttcaggaccaaatgttcaaggcacagatggttctttgtgttccctgctggggtcatgggacttggaaagggatggtaggtagggcttgtgagaagcaggtcttagccataggtgggcagtgactagattttccagcagctgggaagctccagagtacacatccggcaccatgtgaggtatgtgggctttgctggcagggtggacaaggtctgagccacttctgcctctggagttggggaggggggacaggcagaggcctctgcctgccctgccctgctgacctgcccctgcccgttcttcactgaggtatggggctctgctggagcctcttacataatgaacagatgaggctgcagctggggcagccgcccgccctccctcacaccagcatcacgagcctccagtgggcagtccttgggccttgggtggaggccaagcctggttcataaatagggtctccacggtggcctctgctccatctgcccacagtcttccttgctttgcccacgttgctggcctcaggctcagacacctgctctactccaagcaaatggctgctcttccaatgctgtggaccgggctggtcctcttgggtctcttgggatttccacagaccccagcccagggccatgacacagtgcagcccaactttcaacaagacaaggtgagagggtcccctaccccacacccgaggaaacagaaacctcaggtcagagccaggctttctctcacaagagagggtgcgttgggcgctgtcagccatgggagctgtctggaaccgcgctggcacacagcctggttggtccacctgactccgccaggaatgtggctctgatacccactttaccggaagagtagactggggcgagcactgggacaaagacgggagctcaacatcctggggaaggaaggggtcaatgaggcaatgagccagcctactagagagagagaggggcgtggatgctaccagaacctgtgtgtgggaggagtcagagtagggaaggccagcccactagggtctgcccatgaggggcgcatggtgcagacccgggcatccactggtcacagttcctggggcgctggtacagcgcgggcctcgcctccaattcaagctggttccgggagaagaaagagctactgtttatgtgccagacagtggtagctccctccacagaaggcggcctcaacctcacctctaccttcctaaggtgagacaagggggtgtggcaagtttcgggacagaaggccccacaaccctgtctgggggacatcctggggcttgttcccttacatcaggggtaatctacccacaggaaaaaccagtgtgagaccaaggtgatggtactgcagccggcaggggttcccggacagtacacctacaacagcccccgtgagtgagccacttccttatctgggtaaattctgaggtaaatgctggcagactgtgcagccccctgtcccaaaaggtggggataatggtcacaccacaagggtcagtcatccaagaccagacctgattgtgaatctgcctcaggcacacagggctacctctctccagggactttggcctctctgaaacccagccacattcttccaggcccctttcctgtccaaatgaaatttcccagtactctgctgcccaagtgggtcacatacaggcattccccaaatcctacccacatttcatagctcctatccaagtacctctttccatgcctcacctgatctatggattcccaccagaaccctatttccttggccttcctgctatattgtaactcagcctgatgatttcttgagtctaagtgttttctgccctctccccaagattcatggtttggagttagtgttcaggaaggaagctagagattgggtggtggccacccaggggagcacagggaaagaagccaaagcaggggtggaggaggaaggcctgagaccctccccacagagaagcccacaaaggccaccccctccaagcagagggagatagtgatgtgggagccacatgtcttaatcagtgtcatttcttgggttcccagactggggcagcttccactccctctcagtggtagaaaccgactacgatgagtacgcgttcctgttcagcaagggcaccaagggcccaggccaggacttccgcatggccaccctctacagtaggtatcccagcccacaggcccacgcacagggcagatgcctgaggttggaaacagaccaaggcctaacccagaggacagtaacgaaggtgtgtgggggcagggcgagggcttttcacctcctgacaccggccccttctttatctaccaggcagagcccagcttctgaaggaggaactgaaggagaaattcatcacctttagcaaggaccagggcctcacagaggaggacattgttttcctgccccaaccgggtgagggaggctaagctgctgaggagggaattagtgcagattagtgcagcctgtggactggggagagtgtggccgcctactagtccaggggctccaaggaaagaaatggaggtgtcagtctgtcccgacagtacctcgacctgcagccccctttattgggaaccctcttcctggtggacacctcgctgccctgtctgccagccccctagctagggatttaggggcactaacagatggagaaagacaccttttatgttttaaagaacagattggagcaggagtgggatggagtctgaagtgtggggctcagccttggggaggcttcgtaaagtccagggagaagacaaagtcctggtgactgtgggtctaagcctgatactgactacttccctgggcttctttctcaacagataagtgcattcaagagtaaacacaggtgagagaagtcagtcacaggtaacacatggtaagtgccatttactcactcaacataagaccactgagtgctcatgtgaccacggagtgcgggctggggtgggggggatgcagctgcccaaggactgtccaagtgagacagccagagagaaaggacagttccaattccagtggcaggaatagagctgatggccaagggttcatgggagaaggataacagcaatgggaagggaccgccccatgaagcccatcctgcaaaatgagtctccaaggaaccagaatggacaagatcgggaagggactggtggccagggatggacatggcgagtcagagggctggctcctcacctgtgctgttgactgagactctgagaccataggccctggagggataccctaggaggccctggccggaagtgttgtttgggccccactgggctcagggtgctgccctcatcactgatggctcttgttcttctgtgcaggtgatgtggcctcaggactcccgtgctctgtcactcttgagacccaagccctggctccccaaagaccttctccgccctccagctttgccttggtggagaaataaaatccaaagcaagtcagacctcggcttttgtctgtctgtcctccgggccatcactatagccctcttataaatttctcagtatgatgaccagatgggtgtttgtccctgctcaagtcctgagtaggaacagcctgaccaatgcatcaggttcagcgcctactctgcgtagaggggctgcaacctctatgtggtgacataccccaaccaagagagtcacaggtcctgcaagctgccagccacagccaggcctgggctgggctgcggggcgtcagtcacttaaccgctaatcccttagacaagtctacccgtccatccagggagcctcggaccctgtaggttcttcaaggtatggataagaatctctggattaggcaataaagttggaagggcaaaaaggagtcgtttaacagatagagtgggctggagaggctgcctgtacctctgctcctaccccagccctctgaccagagccctagcatcaaaggcaccaaaaccacagatggccacccaattagtcccccttttcttccaaatttccacctgagcagctattcccaagtcctcatctctttccctcctggttcatagtgagcaggtctcaggcccaagcagactacaccaagattcgggtcagcggagagggttgcctctgggaagtcttcctgaagaaaggggatacactatgcctgttctgacacccgagaagtgttaggcagccctcaggcctggaggtcacttgggctacctgcccctgactgctgagttcctcacccctcccactggaaccatgagctgacagggtgtgatgtgggagtgcaagtcaatcagtggtctatcacactgggtgtgtcccaggg

SEQ ID NO: 12 - DNA, 4299 bp, synthetic construct; identical to SEQ ID NO: 11
  source 1..4299: mol_type = other DNA

SEQ ID NO: 13 - DNA, 2929 bp, synthetic construct
  source 1..2929: mol_type = other DNA; note = A sequence fragment between exon 1 and exon 7 of Ptgds gene
cctcaggctcagacacctgctctactccaagcaaatggctgctcttccaatgctgtggaccgggctggtcctcttgggtctcttgggatttccacagaccccagcccagggccatgacacagtgcagcccaactttcaacaagacaaggtgagagggtcccctaccccacacccgaggaaacagaaacctcaggtcagagccaggctttctctcacaagagagggtgcgttgggcgctgtcagccatgggagctgtctggaaccgcgctggcacacagcctggttggtccacctgactccgccaggaatgtggctctgatacccactttaccggaagagtagactggggcgagcactgggacaaagacgggagctcaacatcctggggaaggaaggggtcaatgaggcaatgagccagcctactagagagagagaggggcgtggatgctaccagaacctgtgtgtgggaggagtcagagtagggaaggccagcccactagggtctgcccatgaggggcgcatggtgcagacccgggcatccactggtcacagttcctggggcgctggtacagcgcgggcctcgcctccaattcaagctggttccgggagaagaaagagctactgtttatgtgccagacagtggtagctccctccacagaaggcggcctcaacctcacctctaccttcctaaggtgagacaagggggtgtggcaagtttcgggacagaaggccccacaaccctgtctgggggacatcctggggcttgttcccttacatcaggggtaatctacccacaggaaaaaccagtgtgagaccaaggtgatggtactgcagccggcaggggttcccggacagtacacctacaacagcccccgtgagtgagccacttccttatctgggtaaattctgaggtaaatgctggcagactgtgcagccccctgtcccaaaaggtggggataatggtcacaccacaagggtcagtcatccaagaccagacctgattgtgaatctgcctcaggcacacagggctacctctctccagggactttggcctctctgaaacccagccacattcttccaggcccctttcctgtccaaatgaaatttcccagtactctgctgcccaagtgggtcacatacaggcattccccaaatcctacccacatttcatagctcctatccaagtacctctttccatgcctcacctgatctatggattcccaccagaaccctatttccttggccttcctgctatattgtaactcagcctgatgatttcttgagtctaagtgttttctgccctctccccaagattcatggtttggagttagtgttcaggaaggaagctagagattgggtggtggccacccaggggagcacagggaaagaagccaaagcaggggtggaggaggaaggcctgagaccctccccacagagaagcccacaaaggccaccccctccaagcagagggagatagtgatgtgggagccacatgtcttaatcagtgtcatttcttgggttcccagactggggcagcttccactccctctcagtggtagaaaccgactacgatgagtacgcgttcctgttcagcaagggcaccaagggcccaggccaggacttccgcatggccaccctctacagtaggtatcccagcccacaggcccacgcacagggcagatgcctgaggttggaaacagaccaaggcctaacccagaggacagtaacgaaggtgtgtgggggcagggcgagggcttttcacctcctgacaccggccccttctttatctaccaggcagagcccagcttctgaaggaggaactgaaggagaaattcatcacctttagcaaggaccagggcctcacagaggaggacattgttttcctgccccaaccgggtgagggaggctaagctgctgaggagggaattagtgcagattagtgcagcctgtggactggggagagtgtggccgcctactagtccaggggctccaaggaaagaaatggaggtgtcagtctgtcccgacagtacctcgacctgcagccccctttattgggaaccctcttcctggtggacacctcgctgccctgtctgccagccccctagctagggatttaggggcactaacagatggagaaagacaccttttatgttttaaagaacagattggagcaggagtgggatggagtctgaagtgtggggctcagccttggggaggcttcgtaaagtccagggagaagacaaagtcctggtgactgtgggtctaagcctgatactgactacttccctgggcttctttctcaacagataagtgcattcaagagtaaacacaggtgagagaagtcagtcacaggtaacacatggtaagtgccatttactcactcaacataagaccactgagtgctcatgtgaccacggagtgcgggctggggtgggggggatgcagctgcccaaggactgtccaagtgagacagccagagagaaaggacagttccaattccagtggcaggaatagagctgatggccaagggttcatgggagaaggataacagcaatgggaagggaccgccccatgaagcccatcctgcaaaatgagtctccaaggaaccagaatggacaagatcgggaagggactggtggccagggatggacatggcgagtcagagggctggctcctcacctgtgctgttgactgagactctgagaccataggccctggagggataccctaggaggccctggccggaagtgttgtttgggccccactgggctcagggtgctgccctcatcactgatggctcttgttcttctgtgcaggtgatgtggcctcaggactcccgtgctctgtcactcttgagacccaagccctggctccccaaagaccttctccgccctccagctttgccttggtggagaaataaaatccaaag

(19) United States
(12) Patent Application Publication
(10) Pub. No.: US 2023/0270087 A1
(43) Pub. Date: Aug. 31, 2023

(54) Method for Constructing Ptgds Gene Knockout Rat Model with Spontaneous Kidney Yin Deficiency

(71) Applicant: Shaanxi University of Chinese Medicine, Xianyang (CN)

(72) Inventors: Yanru Liu, Xianyang (CN); Zhishu Tang, Xianyang (CN); Zhongxing Song, Xianyang (CN)

(21) Appl. No.: 18/155,865

(22) Filed: Jan. 18, 2023

(30) Foreign Application Priority Data
Feb. 25, 2022 (CN) 202210191036.7

(51) Int. Cl. A01K 67/027; C12Q 1/6888; C12N 15/873

(52) U.S. Cl. CPC A01K 67/0276; C12Q 1/6888; C12N 15/873; A01K 2227/105; A01K 2267/03; A01K 2217/075

(57) ABSTRACT

The present disclosure belongs to the technical field of bioengineering, and relates to a method for constructing a Ptgds gene knockout rat model with spontaneous kidney yin deficiency. The method includes the following steps: 1) designing target sequences Ptgds-sgRNA1/2; 2) purifying Cas9mRNA and the Ptgds-sgRNA1/2; 3) conducting targeted knockout on a sequence fragment in the Ptgds gene using a CRISPR/Cas9 system; 4) injecting the purified Cas9mRNA, the purified Ptgds-sgRNA1/2, and a Ptgds knockout gene into rat embryos to obtain neonatal rats; 5) conducting genetic identification to select heterozygous rats; and 6) conducting breeding on the heterozygous rats with wild-type rats for multiple generations to obtain the Ptgds gene knockout rat model. In the present disclosure, the method has a high accuracy of gene modification, a targeting specificity, and a short experimental period.

### CROSS REFERENCE TO RELATED APPLICATION

This patent application claims the benefit and priority of Chinese Patent Application No. 202210191036.7 filed with the China National Intellectual Property Administration on Feb. 25, 2022, the disclosure of which is incorporated by reference herein in its entirety as part of the present application.

### REFERENCE TO SEQUENCE LISTING

A computer readable XML file entitled “GWP20220801139”, that was created on Dec. 13, 2022, with a file size of about 28,826 bytes, contains the sequence listing for this application, has been filed with this application, and is hereby incorporated by reference in its entirety.

### TECHNICAL FIELD

The present disclosure belongs to the technical field of bioengineering, and relates to a method for constructing a Ptgds gene knockout rat model with spontaneous kidney yin deficiency.

### BACKGROUND

Perimenopause generally refers to a natural menopausal state after the depletion of follicles in a women's ovaries. It usually shows various symptoms related to the “heart-kidney-reproductive”axis, including symptoms of hot flashes (or vasomotion), chronic renal disease, cardiovascular disease, and neurodegenerative disease, etc. These physical or psychological symptoms are called perimenopausal syndrome. Long-term studies have shown that the perimenopausal syndrome is closely related to development of the chronic kidney disease. From the perspective of traditional Chinese medicine dialectics, the perimenopausal syndrome is due to kidney deficiency, mostly kidney yin deficiency, typically showing symptoms of hot flashes, obesity, and central degeneration. Perimenopausal syndrome is due to the fact that from the age of 49, kidney qi gradually becomes deficient in women, and the effect of menstruation also disappears. Conception Vessel and Thoroughfare Vessel tend to be feeble, and the essence and blood for nourishing yin decrease, resulting in symptoms of yin deficiency, amenorrhea, and gradual infertility. Therefore, “Su Wen” recorded: “At the age of 49, the Conception Vessel and the Thoroughfare Vessel is feeble, the menstruation is exhausted to cause amenorrhea, vagina atrophy, female characteristics are destroyed and women become infertility”.

Existing studies have shown that estrogen depletion attenuates adipocyte transport in menopausal rat models. Prostaglandin D2 synthase (Ptgds) has increased expression in kidney and decreased expression in uterus and hypothalamus, thereby weakening renal lipid metabolism. As a result, typical symptoms of kidney yin deficiency in menopause appear in a cascade, including renal metabolic disorders such as hot flashes, weight gain, elevated blood glucose, and abnormal lipid metabolism. Experimental data further confirm that upstream estrogen receptor β (ERβ) depletion activates the overexpression of Ptgds in renal, leading to an imbalance in renal lipid metabolism and the reduced transport of Ptgds to the hypothalamus. Moreover, this factor may continue to accelerate the degeneration of central nervous system function, and typical central degenerative symptoms such as decreased learning ability and memory decline appear in the experiment.

Ptgds is a non-glutathione-independent and lipocalin-type PGD synthetase. As a monomeric member of the lipocalin family, Ptgds, is an approximately 26 kDa protein composed of 189 amino acid residues, includes a signal sequence (aa 1-24), and a lipocalin region that serves as both a catalytic site and a hydrophobic molecule transporter (aa 40-187). Ptgds is mainly localized in the Golgi apparatus and nuclear membrane of cells or secreted to extracellular regions, and are mainly expressed in the brain, central nervous system, prostate, uterus, and kidney. Ptgds has the function of catalyze the synthesis of prostaglandin D2 (PGD2) and transport the lipophilic substances. Ptgds can also catalyze the conversion of PGH2 to PGD2, thereby affecting sleep and body temperature. In addition, activation of Ptgds can affect lipid metabolic shifts, such as eicosanes metabolism in the arachidonic acid, α-linolenic acid (ala), and cyclooxygenase (cox) pathways. Urine-secreted Ptgds is synthesized in glomeruli and glomerular rings. Due to a low molecular weight and anionic properties, Ptgds can pass through the glomerular capillary wall more easily than serum albumin to more accurately reflect changes in glomerular permeability, which is the key marker for the diagnosis of kidney diseases.

In order to conduct related researches on menopausal kidney yin deficiency, animal models are generally prepared by surgically removing both ovaries of female rats. This is because the rat has a well-developed pituitary-adrenal function and a sensitive stress response, such that the rat is especially suitable for stress response and endocrine experimental researches of pituitary, epinephrine, and ovary, etc. However, the construction of rat models have several problems, such as the construction takes a long time and the model animals are difficult to be raised. Therefore, it is urgently needed to find an efficient and stable method to construct the animal models. Currently, gene knockout models are commonly constructed and bred to solve the above problem. However, there are only reports on mouse models with Ptgds gene knockout (Ptgds−/−) in the prior art, and there is no relevant report on rat models with the Ptgds−/−.

### SUMMARY

Aiming at the technical problems in the existing construction of gene knockout rat models, the present disclosure provides a method for constructing a Ptgds gene knockout rat model with spontaneous kidney yin deficiency. The method has high accuracy of gene modification, targeting specificity, and a short experimental period. Therefore, a reliable and stable genetically-engineered model is developed to lay the foundation for a therapeutic effect of menopausal syndrome-related diseases.

To achieve the above objective, the present disclosure adopts the following technical solution:

The present disclosure provides a method for constructing a Ptgds gene knockout rat model with spontaneous kidney yin, including the following steps:

1) designing two target sequences Ptgds-sgRNA1 and Ptgds-sgRNA2 at a Ptgds gene locus;

2) obtaining purified Cas9mRNA, purified Ptgds-sgRNA1, and purified Ptgds-sgRNA2 by in vitro transcription;

3) conducting targeted knockout on a 2,944 bp sequence fragment in the Ptgds gene using a CRISPR/Cas9 system to obtain a Ptgds knockout gene;

4) injecting the purified Cas9mRNA, the purified Ptgds-sgRNA1, the purified Ptgds-sgRNA2, and the Ptgds knockout gene into rat embryos, and transplanting the embryos into fallopian tubes of surrogate recipient rats to obtain neonatal rats;

5) conducting gene identification on the neonatal rats to select heterozygous rats; and

6) conducting breeding on the heterozygous rats with wild-type rats for multiple generations, and subjecting offspring rats obtained from each generation to gene identification until obtaining homozygous rats,that is the Ptgds gene knockout rat model.

Further, in step 1), the Ptgds-sgRNA1 has a nucleotide sequence set forth in SEQ ID NO: 1; and the Ptgds-sgRNA2 has a nucleotide sequence set forth in SEQ ID NO: 3.

Further, in step 3), the sequence fragment includes an intron sequence fragment and an exon sequence fragment.

Further, in step 3), the intron sequence fragment has a nucleotide sequence set forth in SEQ ID NO: 5; and the exon sequence fragment has a nucleotide sequence set forth in SEQ ID NO: 6.

Further, the gene identification in step 5) and step 6) includes the following steps:

S1) extracting a genomic DNA from the neonatal rat;

S2) conducting PCR amplification with specific primers using the genomic DNA as a template to obtain an amplification product;

S3) conducting electrophoresis detection on the amplification product using agarose gel; and

S4) identifying the heterozygous rats or the homozygous rats according to an electrophoresis result.

Further, in step S2), the specific primers include primers of Ptgds-L-S, Ptgds-L-A, Ptgds-R-S, and Ptgds-R-A, with nucleotide sequences set forth in SEQ ID NO: 7, SEQ ID NO: 8, SEQ ID NO: 9, and SEQ ID NO: 10, respectively.

Further, in step S2), a reaction system of the PCR amplification includes: 2.5 μl of a template DNA at 500 ng/μl, 2.5 μl of each of the Ptgds-L-S, the Ptgds-L-A, the Ptgds-R-S, and the Ptgds-R-A that are at 10 μmol/L, 5 μl of a 10× buffer, 5 μl of dNTP at 2.5 mmol/L, 0.5 μl of Eazy-taq, and supplementing to 50 μl with water; and

the PCR amplification includes: pre-denaturation at 98° C. for 2 min; denaturation at 98° C. for 20 s×30; annealing at 55° C. for 20 s×30; extension at 72° C. for 10 s×30; terminal extension at 72° C. for 5 min; and cooling at 16° C. for 2 min.

Further, step 6) specifically includes the following steps:

6.1) using the heterozygous rats selected in step 5) as F0-generation heterozygous rats, caging with the wild-type rats, conducting gene identification on obtained offspring I, and selecting heterozygous rats from the offspring I as F1-generation rats;

6.2) caging the F1-generation rats with the wild-type rats, conducting gene identification on obtained offspring II, and selecting heterozygous rats from the offspring II as F2-generation rats; and

6.3) using rats generated by conducting self-breeding within the group of F2-generation rats as F3-generation rats, and conducting gene identification to select homozygous rats in F3-generation as the Ptgds gene knockout rat model.

The present disclosure has the following beneficial effects:

1. The present disclosure provides a CRISPR/Cas9 technology to construct a Ptgds gene knockout rat model, which has the advantage of high gene modification accuracy, specific targeting, short experimental period, and no species restriction.

2. In the present disclosure, during construction of the gene knockout rat model, nicks are made on exon 1 of the Ptgds gene spliceosome Ptgds-201 and the non-coding region of exon 7, the two nicks are directly ligated through an NHEJ repair pathway, and the sequence between the two nicks (namely the entire coding sequence) is deleted, so as to knock out the Ptgds gene. The purified Cas9mRNA and purified sgRNA obtained by in vitro transcription are injected into SD rat embryos. After injection, the embryos are transplanted into the fallopian tubes of surrogate recipient rats. And the Ptgds gene knockout rat model is obtained by breeding the embryos. The present disclosure provides a reliable and stable genetic engineering model for further researches on an influence of the spontaneous menopausal kidney yin deficiency in rats, and lays the foundation for clarifying a therapeutic effect of menopausal syndrome-related diseases and the like.

3. The present disclosure is based on use of the CRISPR/Cas9 technology in targeted knockout of the Ptgds gene in rats to construct a Ptgds gene knockout (Ptgds−/−) rat model, which has spontaneous kidney yin deficiency symptoms; through reproduction, PCR identification, and pathological index detection, the model can be further applied to pharmacodynamic evaluation. The model can provide a reliable and stable genetic engineering model for the study of symptoms of kidney yin deficiency during perimenopausal, and lay a foundation for exploration of perimenopausal syndrome mechanism and evaluation of related drug treatment effects.

### BRIEF DESCRIPTION OF THE DRAWINGS

FIG. 1 shows a schematic diagram of a Cas9 targeted knockout strategy of a Ptgds gene in SD rats;

FIG. 2 shows results L-A&L-S of PCR identification of phenotypic of Ptgds gene knockout rats;

FIG. 3 shows results R-A/L-S of PCR identification of phenotypic of Ptgds gene knockout rats;

FIG. 4 shows a schematic diagram of reproduction and generations of a Ptgds gene knockout rat model;

FIG. 5 shows 8-month-old body weight results of the Ptgds gene knockout rats;

FIG. 6 shows organ index results of the Ptgds gene knockout rats;

FIG. 7 shows measurement results of a tail temperature of the Ptgds gene knockout rats;

FIG. 8 shows infrared thermal imaging results of the Ptgds gene knockout rats;

FIG. 9 shows serum biochemical assay results of the Ptgds gene knockout rats;

FIG. 10 shows ELISA results of a kidney function of the Ptgds gene knockout rats;

FIGS. 11A-D shows ELISA results of other gonad-related organ functions of the Ptgds gene knockout rats; and

FIG. 12 shows results of a Morris water maze test of the Ptgds gene knockout rats.

### DETAILED DESCRIPTION OF THE EMBODIMENTS

The method for constructing model in present disclosure is further described in detail below with reference to the accompanying drawings and examples.

The present disclosure provides a method for constructing a Ptgds gene knockout rat model with spontaneous kidney yin deficiency, including the following steps:

1) two target sequences Ptgds-sgRNA1 and Ptgds-sgRNA2 at a Ptgds gene locus is designed.

2) purified Cas9mRNA, purified Ptgds-sgRNA1, and purified Ptgds-sgRNA2 were obtained by in vitro transcription.

3) CRISPR/Cas9 system to conduct targeted knockout on a 2,944 bp sequence fragment in the Ptgds gene to obtain a Ptgds knockout gene.

4) The purified Cas9mRNA, the purified Ptgds-sgRNA1, the purified Ptgds-sgRNA2, and the Ptgds knockout gene are injected into rat embryos, and the embryos are transplanted into fallopian tubes of surrogate recipient rats to obtain neonatal rats.

5) Gene identification was conducted on the neonatal rats to select heterozygous rats.

6) Multi-generation reproduction between heterozygous rats and wild-type rats was conducted, and offspring rats obtained from each generation are subjected to gene identification until homozygous rats are genetically identified as the Ptgds gene knockout rat model.

In the present disclosure, in step 1), the Ptgds-sgRNA1 has a nucleotide sequence set forth in SEQ ID NO: 1; and the Ptgds-sgRNA2 has a nucleotide sequence set forth in SEQ ID NO: 3.

In the present disclosure, in step 3), the sequence fragment includes an intron sequence fragment and an exon sequence fragment.

In the present disclosure, in step 3), the intron sequence fragment has a nucleotide sequence set forth in SEQ ID NO: 5; and the exon sequence fragment has a nucleotide sequence set forth in SEQ ID NO: 6.

In the present disclosure, the gene identification in step 5) and step 6) includes the following steps:

S1) a genomic DNA is extracted from the neonatal rat.

S2) PCR amplification was conducted with specific primers using the genomic DNA as a template to obtain an amplification product.

S3) electrophoresis detection was conducted on the amplification product using agarose gel.

S4) the heterozygous rats or the homozygous rats were identified according to an electrophoresis result.

In the present disclosure, in step S2), the specific primers include primers of Ptgds-L-S, Ptgds-L-A, Ptgds-R-S, and Ptgds-R-A, with nucleotide sequences set forth in SEQ ID NO: 7, SEQ ID NO: 8, SEQ ID NO: 9, and SEQ ID NO: 10, respectively.

In the present disclosure, in step S2), a reaction system of the PCR amplification includes: 2.5 μl of a template DNA at 500 ng/μl, 2.5 μl of each of the Ptgds-L-S, the Ptgds-L-A, the Ptgds-R-S, and the Ptgds-R-A that are at 10 μmol/L, 5 μl of a 10× buffer, 5 μl of dNTP at 2.5 mmol/L, 0.5 μl of Eazy-taq, and supplementing to 50 μl with water; and

the PCR amplification includes: pre-denaturation at 98° C. for 2 min; denaturation at 98° C. for 20 sec×30; annealing at 55° C. for 20 sec×30; extension at 72° C. for 10 sec×30; terminal extension at 72° C. for 5 min; and cooling at 16° C. for 2 min.

In the present disclosure, step 6) specifically includes the following steps:

6.1) the heterozygous rats selected in step 5) are used as F0-generation heterozygous rats, caged with the wild-type rats, then a gene identification is conducted on obtained offspring I, and the heterozygous rats are selected from the offspring I as F1-generation rats;

6.2) the F1-generation rats with the wild-type rats are caged, then a genetic identification is conducted on obtained offspring II, and the heterozygous rats are selected from the offspring II as F2-generation rats; and

6.3) rats generated by conducting self-breeding within the group of F2-generation rats are used as F3-generation rats, and a genetic identification is conducted to select homozygous rats as the Ptgds gene knockout rat model.

### EXAMPLE

The following described a method for constructing a rat model by a specific example.

### 1. Ptgds Gene Information of SD Rats

In this example, a constructed rat Ptgds gene was located on chromosome 3 of the SD rats, with only one transcript: Ptgds-201.

Therefore, the model was constructed with the Ptgds-201 transcript as an object.

### 2. Ptgds Protein Information of SD Rats

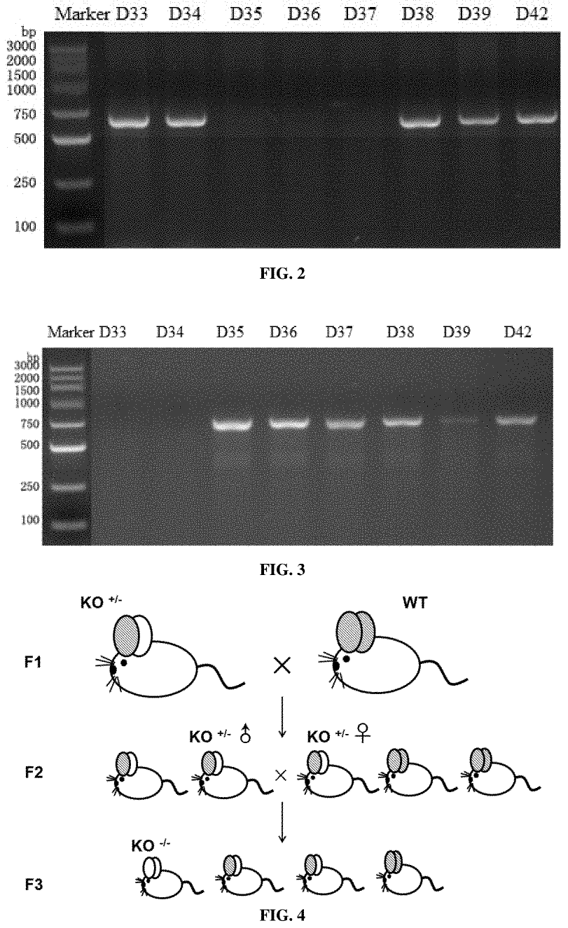

As shown in FIG. 2 and FIG. 3, a Ptgds protein domain and a Ptgds protein expression profile of the SD rats were provided in this example.

### 3. Construction Ideas of Gene Knockout Rat Model

A specific sgRNA (single-guide RNA) of the Ptgds gene of rat mediated the cleavage of DNA by a Cas9 nuclease to produce specific DSBs (Double-Stranded Breaks), and an entire encoding sequence of the gene was deleted.

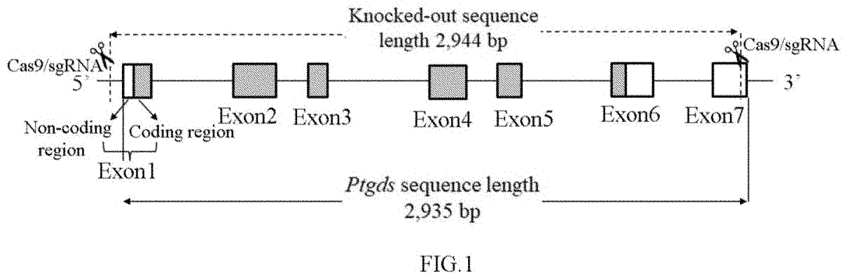

Referring to FIG. 1, when being implemented, nicks were made on exon 1 of a spliceosome Ptgds-201 and a non-coding region of exon 7; through an NHEJ (Non-homologous end Joining) repair pathway, the two nicks were directly ligated, and a sequence between the two nicks (namely all encoding sequences) was deleted; targets ending with NGG were designed on both sides of Ptgds exon1-exon7, and a sequence between the two targets was expected to be knocked out, thereby to achieve the knockout of the Ptgds gene.

### (1) Design of a Guide RNA (gRNA)

In this example, two target sequences Ptgds-sgRNA1 and Ptgds-sgRNA2 were designed at a Ptgds gene locus;

Ptgds-sgRNA1:
(SEQ ID NO: 1)

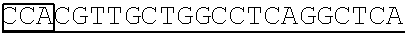

Reverse complement (Re):
(SEQ ID NO: 2)

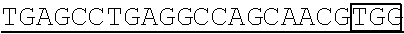

Ptgds-sgRNA2:
(SEQ ID NO: 3)

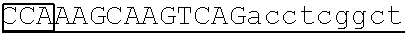

Reverse complement (Re):
(SEQ ID NO: 4)

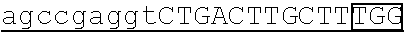

In the above sequence, several genes in the box were PAM recognition sites, which were used to recognize a target sequence of a cleavage position; and a rat model was formed by injecting Ptgds-sgRNA1, Ptgds-sgRNA2 and CAS9 proteins into fertilized eggs to delete the PTGDS gene.

### (2) Gene Sequence and Guide RNA Information

With a CRISPR/Cas9 system, an intron sequence fragment (15 bp, lowercase, bold and underline) associated with the Ptgds gene (SEQ ID NO:6, 2,935 bp, uppercase) and a sequence fragment (SEQ ID NO:13, 2,929 bp, uppercase, bold and underlined) between exon 1 and exon 7 of the Ptgds gene, that is, a sequence fragment with a total length of 2,944 bp was subjected to targeted knockout.

(SEQ ID NO: 11)
tagcctttcaggaccaaatgttcaaggcacagatggttctttgtgttccctgctggggtcatgggacttggaaagggatggtaggtag
ggcttgtgagaagcaggtcttagccataggtgggcagtgactagattttccagcagctgggaagctccagagtacacatccggcaccatgtgag
gtatgtgggctttgctggcagggtggacaaggtctgagccacttctgcctctggagttggggaggggggacaggcagaggcctctgcctgccct
gccctgctgacctgcccctgcccgttcttcactgaggtatggggctctgctggagcctcttacataatgaacagatgaggctgcagctggggcagc
cgcccgccctccctcacaccagcatcacgagcctccagtgggcagtccttgggccttgggtggaggccaagcctggttcataaatagggtctcc
acggtggcctctgctccatctgcccacagtcttccttgctttgcccacgttgctggCCTCAGGCTCAGACACCTGCTCTA
CTCCAAGCAAATGGCTGCTCTTCCAATGCTGTGGACCGGGCTGGTCCTCTTGGGTC
TCTTGGGATTTCCACAGACCCCAGCCCAGGGCCATGACACAGTGCAGCCCAACTTT
CAACAAGACAAGGTGAGAGGGTCCCCTACCCCACACCCGAGGAAACAGAAACCTC
AGGTCAGAGCCAGGCTTTCTCTCACAAGAGAGGGTGCGTTGGGCGCTGTCAGCCA
TGGGAGCTGTCTGGAACCGCGCTGGCACACAGCCTGGTTGGTCCACCTGACTCCG
CCAGGAATGTGGCTCTGATACCCACTTTACCGGAAGAGTAGACTGGGGCGAGCACT
GGGACAAAGACGGGAGCTCAACATCCTGGGGAAGGAAGGGGTCAATGAGGCAATG
AGCCAGCCTACTAGAGAGAGAGAGGGGCGTGGATGCTACCAGAACCTGTGTGTGG
GAGGAGTCAGAGTAGGGAAGGCCAGCCCACTAGGGTCTGCCCATGAGGGGCGCAT
GGTGCAGACCCGGGCATCCACTGGTCACAGTTCCTGGGGCGCTGGTACAGCGCGG
GCCTCGCCTCCAATTCAAGCTGGTTCCGGGAGAAGAAAGAGCTACTGTTTATGTGC
CAGACAGTGGTAGCTCCCTCCACAGAAGGCGGCCTCAACCTCACCTCTACCTTCCT
AAGGTGAGACAAGGGGGTGTGGCAAGTTTCGGGACAGAAGGCCCCACAACCCTGT
CTGGGGGACATCCTGGGGCTTGTTCCCTTACATCAGGGGTAATCTACCCACAGGAA
AAACCAGTGTGAGACCAAGGTGATGGTACTGCAGCCGGCAGGGGTTCCCGGACAG
TACACCTACAACAGCCCCCGTGAGTGAGCCACTTCCTTATCTGGGTAAATTCTGAG
GTAAATGCTGGCAGACTGTGCAGCCCCCTGTCCCAAAAGGTGGGGATAATGGTCAC
ACCACAAGGGTCAGTCATCCAAGACCAGACCTGATTGTGAATCTGCCTCAGGCACA
CAGGGCTACCTCTCTCCAGGGACTTTGGCCTCTCTGAAACCCAGCCACATTCTTCC
AGGCCCCTTTCCTGTCCAAATGAAATTTCCCAGTACTCTGCTGCCCAAGTGGGTCA
CATACAGGCATTCCCCAAATCCTACCCACATTTCATAGCTCCTATCCAAGTACCTCTT
TCCATGCCTCACCTGATCTATGGATTCCCACCAGAACCCTATTTCCTTGGCCTTCCT
GCTATATTGTAACTCAGCCTGATGATTTCTTGAGTCTAAGTGTTTTCTGCCCTCTCCC
CAAGATTCATGGTTTGGAGTTAGTGTTCAGGAAGGAAGCTAGAGATTGGGTGGTGG
CCACCCAGGGGAGCACAGGGAAAGAAGCCAAAGCAGGGGTGGAGGAGGAAGGCC
TGAGACCCTCCCCACAGAGAAGCCCACAAAGGCCACCCCCTCCAAGCAGAGGGAG
ATAGTGATGTGGGAGCCACATGTCTTAATCAGTGTCATTTCTTGGGTTCCCAGACTG
GGGCAGCTTCCACTCCCTCTCAGTGGTAGAAACCGACTACGATGAGTACGCGTTCC
TGTTCAGCAAGGGCACCAAGGGCCCAGGCCAGGACTTCCGCATGGCCACCCTCTA
CAGTAGGTATCCCAGCCCACAGGCCCACGCACAGGGCAGATGCCTGAGGTTGGAA
ACAGACCAAGGCCTAACCCAGAGGACAGTAACGAAGGTGTGTGGGGGCAGGGCGA
GGGCTTTTCACCTCCTGACACCGGCCCCTTCTTTATCTACCAGGCAGAGCCCAGCT
TCTGAAGGAGGAACTGAAGGAGAAATTCATCACCTTTAGCAAGGACCAGGGCCTCA
CAGAGGAGGACATTGTTTTCCTGCCCCAACCGGGTGAGGGAGGCTAAGCTGCTGA
GGAGGGAATTAGTGCAGATTAGTGCAGCCTGTGGACTGGGGAGAGTGTGGCCGCC
TACTAGTCCAGGGGCTCCAAGGAAAGAAATGGAGGTGTCAGTCTGTCCCGACAGTA
CCTCGACCTGCAGCCCCCTTTATTGGGAACCCTCTTCCTGGTGGACACCTCGCTGC
CCTGTCTGCCAGCCCCCTAGCTAGGGATTTAGGGGCACTAACAGATGGAGAAAGAC
ACCTTTTATGTTTTAAAGAACAGATTGGAGCAGGAGTGGGATGGAGTCTGAAGTGT
GGGGCTCAGCCTTGGGGAGGCTTCGTAAAGTCCAGGGAGAAGACAAAGTCCTGGT
GACTGTGGGTCTAAGCCTGATACTGACTACTTCCCTGGGCTTCTTTCTCAACAGATA
AGTGCATTCAAGAGTAAACACAGGTGAGAGAAGTCAGTCACAGGTAACACATGGTA
AGTGCCATTTACTCACTCAACATAAGACCACTGAGTGCTCATGTGACCACGGAGTG
CGGGCTGGGGTGGGGGGGATGCAGCTGCCCAAGGACTGTCCAAGTGAGACAGCCA
GAGAGAAAGGACAGTTCCAATTCCAGTGGCAGGAATAGAGCTGATGGCCAAGGGT
TCATGGGAGAAGGATAACAGCAATGGGAAGGGACCGCCCCATGAAGCCCATCCTGC
AAAATGAGTCTCCAAGGAACCAGAATGGACAAGATCGGGAAGGGACTGGTGGCCA
GGGATGGACATGGCGAGTCAGAGGGCTGGCTCCTCACCTGTGCTGTTGACTGAGA
CTCTGAGACCATAGGCCCTGGAGGGATACCCTAGGAGGCCCTGGCCGGAAGTGTT
GTTTGGGCCCCACTGGGCTCAGGGTGCTGCCCTCATCACTGATGGCTCTTGTTCTT
CTGTGCAGGTGATGTGGCCTCAGGACTCCCGTGCTCTGTCACTCTTGAGACCCAAG
CCCTGGCTCCCCAAAGACCTTCTCCGCCCTCCAGCTTTGCCTTGGTGGAGAAATAA
AATCCAAAGCAAGTCagacctcggcttttgtctgtctgtcctccgggccatcactatagccctcttataaatttctcagtatgatgacca
gatgggtgtttgtccctgctcaagtcctgagtaggaacagcctgaccaatgcatcaggttcagcgcctactctgcgtagaggggctgcaacctctat
gtggtgacataccccaaccaagagagtcacaggtcctgcaagctgccagccacagccaggcctgggctgggctgcggggcgtcagtcacttaa
ccgctaatcccttagacaagtctacccgtccatccagggagcctcggaccctgtaggttcttcaaggtatggataagaatctctggattaggcaat
aaagttggaagggcaaaaaggagtcgtttaacagatagagtgggctggagaggctgcctgtacctctgctcctaccccagccctctgaccaga
gccctagcatcaaaggcaccaaaaccacagatggccacccaattagtcccccttttcttccaaatttccacctgagcagctattcccaagtcctcatc
tctttccctcctggttcatagtgagcaggtctcaggcccaagcagactacaccaagattcgggtcagcggagagggttgcctctgggaagtcttcct
gaagaaaggggatacactatgcctgttctgacacccgagaagtgttaggcagccctcaggcctggaggtcacttgggctacctgcccctgactgc
tgagttcctcacccctcccactggaaccatgagctgacagggtgtgatgtgggagtgcaagtcaatcagtggtctatcacactgggtgtgtcccag
gg.

### 4. Design, In Vitro Transcription and Purification of gRNA

gRNA was designed through sequence alignment; since it was a large fragment deletion gene, two gRNAs were designed for each target site to be cut, but only one gRNA was used for each site, and the gRNA with a higher off-target score was preferentially used, that was, the sgRNA with a low off-target probability was selected; another gRNA was alternative, and the gene was knocked out using the alternative gRNA when the higher-scoring gRNAs failed to work.

The DNA fragment of sgRNA was amplified by PCR with a sgRNA-Vector as a template, and then recovered by gel as a template for in vitro transcription of sgRNA, and then recovered by the in vitro transcription of sgRNA and purification, and stored in a −80° C. refrigerator for later use.

### 5. Microinjection

The purified sgRNA and the Cas9-mRNA were injected into embryos of SD rat, and the embryos were transplanted into fallopian tubes of surrogate recipient rats to obtain neonatal rats.

### 6. Breeding of Gene Knockout Rats

Neonatal rats were obtained 21 d after embryo transfer, and genotype identification was completed about 2 weeks after birth.

### (1) Genotype Identification of Rats

Genotyping of neonatal rats was analyzed by genotype identification. Rats around 14 d after birth were numbered by toe clipping method and subjected to genotype identification.

### (2) Method for the Genotype Identification of Rats

Step 1: Extraction of genomic DNA

(1) Digestion

About a week after the rat was born, 0.5 cm of the rat toe was cut and placed in a 1.5 ml EP tube. After slight centrifugation, 500 μl and 0.5 μl of proteinase K (concentration: 20 mg/ml, dissolved in pH 7.4, 20 mmol/L Tris and 1 mmol/CaCl₂, stored in a 50% glycerol buffer solution at −20° C.), were mixed well and digested in a 55° C. water bath overnight;

A formula of lysis solution included: 100 mmol/L Tris at pH 8.0, 5 mmol/L EDTA at pH 8.0, 0.5% SDS, and 1.17 g/100 ml NaCl.

### (2) DNA Extraction by Isopropanol Precipitation

1) The centrifuge tube was removed from the water bath and allowed to stand at room temperature for 10 min to 15 min, such that the sample was cooled to room temperature, the centrifuge tube was inverted to mix well, and centrifuged at 13,000 rpm at room temperature for 15 min.

2) 400 μl of a supernatant was pipetted into another new centrifuge tube. An equal volume of isopropanol was added, and the tube was turned up and down gently to mix thoroughly. At this time, a white flocculent precipitate appeared, and the tube was centrifuged at 12,000 rpm for 10 min at room temperature, and the supernatant was discarded.

3) The centrifuge tube was rinsed with 700 μl of cold 75% ethanol, and gently turned up and down to mix well. Centrifugation was conducted at 12,000 rpm for 5 min at room temperature, and all the supernatant was removed by suction.

4) The centrifuge tube was inverted on absorbent paper to blot dry the ethanol. After air-drying, the DNA was dissolved with 50 μl of sterile ddH₂O at 55° C. for 2 h (if not being used immediately, the DNA was stored at −20° C.).

5) The concentration of DNA was detected, and 100 ng to 200 ng of the DNA was used as a PCR template.

Step 2: PCR amplification was conducted with specific primers using the genomic DNA as a template to obtain an amplification product; and

forward and reverse PCR primers were designed for about 200 bp to 300 bp of upstream and downstream regions of the target.

(1) The primer information was shown in Table 1.

TABLE 1
Specific primer results

| | | | Primer |
|---|---|---|---|
| | | Tm | length |
| Primer | 5′---3′ | (° C.) | (bp) |
| Ptgds-L-S (lowercase, bold, and | GCAGGTCTTAGCCATAGGTG | 55,+DMSO | 609 |
| shade of gray) | (SEQ ID NO: 7) | | |
| Ptgds-L-A (uppercase, bold, and | TTTCTGTTTCCTCGGGTG | | |
| shade of gray) | (SEQ ID NO: 8) | | |
| Ptgds-R-S | GATGGCTCTTGTTCTTCTGTG | 55 | 516 |
| | (SEQ ID NO: 9) | | |
| Ptgds-R-A | CCTTCCAACTTTATTGCCTAAT | | |
| | (SEQ ID NO: 10) | | |

(SEQ ID NO: 12)
tagcctttcaggaccaaatgttcaaggcacagatggttctttgtgttccctgctggggtcatgggacttggaaagggatggtaggtagg
gcttgtgagaagcaggtcttagccataggtgggcagtgactagattttccagcagctgggaagctccagagtacacatccggcaccatgtgagg
tatgtgggctttgctggcagggtggacaaggtctgagccacttctgcctctggagttggggaggggggacaggcagaggcctctgcctgccctg
ccctgctgacctgcccctgcccgttcttcactgaggtatggggctctgctggagcctcttacataatgaacagatgaggctgcagctggggcagcc
gcccgccctccctcacaccagcatcacgagcctccagtgggcagtccttgggccttgggtggaggccaagcctggttcataaatagggtctcca
cggtggcctctgctccatctgcccacagtcttccttgctttgcccacgttgctggCCTCAGGCTCAGACACCTGCTCTAC
TCCAAGCAAATGGCTGCTCTTCCAATGCTGTGGACCGGGCTGGTCCTCTTGGGTCT
CTTGGGATTTCCACAGACCCCAGCCCAGGGCCATGACACAGTGCAGCCCAACTTTC
AACAAGACAAGGTGAGAGGGTCCCCTACCCCACACCCGAGGAAACAGAAACCTCA
GGTCAGAGCCAGGCTTTCTCTCACAAGAGAGGGTGCGTTGGGCGCTGTCAGCCAT
GGGAGCTGTCTGGAACCGCGCTGGCACACAGCCTGGTTGGTCCACCTGACTCCGC
CAGGAATGTGGCTCTGATACCCACTTTACCGGAAGAGTAGACTGGGGCGAGCACT
GGGACAAAGACGGGAGCTCAACATCCTGGGGAAGGAAGGGGTCAATGAGGCAAT
GAGCCAGCCTACTAGAGAGAGAGAGGGGCGTGGATGCTACCAGAACCTGTGTGTG
GGAGGAGTCAGAGTAGGGAAGGCCAGCCCACTAGGGTCTGCCCATGAGGGGCGC
ATGGTGCAGACCCGGGCATCCACTGGTCACAGTTCCTGGGGCGCTGGTACAGCGC
GGGCCTCGCCTCCAATTCAAGCTGGTTCCGGGAGAAGAAAGAGCTACTGTTTATGT
GCCAGACAGTGGTAGCTCCCTCCACAGAAGGCGGCCTCAACCTCACCTCTACCTTC
CTAAGGTGAGACAAGGGGGTGTGGCAAGTTTCGGGACAGAAGGCCCCACAACCCT
GTCTGGGGGACATCCTGGGGCTTGTTCCCTTACATCAGGGGTAATCTACCCACAG
GAAAAACCAGTGTGAGACCAAGGTGATGGTACTGCAGCCGGCAGGGGTTCCCGGA
CAGTACACCTACAACAGCCCCCGTGAGTGAGCCACTTCCTTATCTGGGTAAATTCT
GAGGTAAATGCTGGCAGACTGTGCAGCCCCCTGTCCCAAAAGGTGGGGATAATGG
TCACACCACAAGGGTCAGTCATCCAAGACCAGACCTGATTGTGAATCTGCCTCAGG
CACACAGGGCTACCTCTCTCCAGGGACTTTGGCCTCTCTGAAACCCAGCCACATTC
TTCCAGGCCCCTTTCCTGTCCAAATGAAATTTCCCAGTACTCTGCTGCCCAAGTGG
GTCACATACAGGCATTCCCCAAATCCTACCCACATTTCATAGCTCCTATCCAAGTA
CCTCTTTCCATGCCTCACCTGATCTATGGATTCCCACCAGAACCCTATTTCCTTGGC
CTTCCTGCTATATTGTAACTCAGCCTGATGATTTCTTGAGTCTAAGTGTTTTCTGCC
CTCTCCCCAAGATTCATGGTTTGGAGTTAGTGTTCAGGAAGGAAGCTAGAGATTGG
GTGGTGGCCACCCAGGGGAGCACAGGGAAAGAAGCCAAAGCAGGGGTGGAGGAG
GAAGGCCTGAGACCCTCCCCACAGAGAAGCCCACAAAGGCCACCCCCTCCAAGCA
GAGGGAGATAGTGATGTGGGAGCCACATGTCTTAATCAGTGTCATTTCTTGGGTTC
CCAGACTGGGGCAGCTTCCACTCCCTCTCAGTGGTAGAAACCGACTACGATGAGT
ACGCGTTCCTGTTCAGCAAGGGCACCAAGGGCCCAGGCCAGGACTTCCGCATGGC
CACCCTCTACAGTAGGTATCCCAGCCCACAGGCCCACGCACAGGGCAGATGCCTG
AGGTTGGAAACAGACCAAGGCCTAACCCAGAGGACAGTAACGAAGGTGTGTGGGG
GCAGGGCGAGGGCTTTTCACCTCCTGACACCGGCCCCTTCTTTATCTACCAGGCAG
AGCCCAGCTTCTGAAGGAGGAACTGAAGGAGAAATTCATCACCTTTAGCAAGGAC
CAGGGCCTCACAGAGGAGGACATTGTTTTCCTGCCCCAACCGGGTGAGGGAGGCT
AAGCTGCTGAGGAGGGAATTAGTGCAGATTAGTGCAGCCTGTGGACTGGGGAGAG
TGTGGCCGCCTACTAGTCCAGGGGCTCCAAGGAAAGAAATGGAGGTGTCAGTCTG
TCCCGACAGTACCTCGACCTGCAGCCCCCTTTATTGGGAACCCTCTTCCTGGTGGA
CACCTCGCTGCCCTGTCTGCCAGCCCCCTAGCTAGGGATTTAGGGGCACTAACAG
ATGGAGAAAGACACCTTTTATGTTTTAAAGAACAGATTGGAGCAGGAGTGGGATG
GAGTCTGAAGTGTGGGGCTCAGCCTTGGGGAGGCTTCGTAAAGTCCAGGGAGAAG
ACAAAGTCCTGGTGACTGTGGGTCTAAGCCTGATACTGACTACTTCCCTGGGCTTC
TTTCTCAACAGATAAGTGCATTCAAGAGTAAACACAGGTGAGAGAAGTCAGTCACA
GGTAACACATGGTAAGTGCCATTTACTCACTCAACATAAGACCACTGAGTGCTCAT
GTGACCACGGAGTGCGGGCTGGGGTGGGGGGGATGCAGCTGCCCAAGGACTGTC
CAAGTGAGACAGCCAGAGAGAAAGGACAGTTCCAATTCCAGTGGCAGGAATAGAG
CTGATGGCCAAGGGTTCATGGGAGAAGGATAACAGCAATGGGAAGGGACCGCCCC
ATGAAGCCCATCCTGCAAAATGAGTCTCCAAGGAACCAGAATGGACAAGATCGGG
AAGGGACTGGTGGCCAGGGATGGACATGGCGAGTCAGAGGGCTGGCTCCTCACCT
GTGCTGTTGACTGAGACTCTGAGACCATAGGCCCTGGAGGGATACCCTAGGAGGC
CCTGGCCGGAAGTGTTGTTTGGGCCCCACTGGGCTCAGGGTGCTGCCCTCATCAC
TGATGGCTCTTGTTCTTCTGTGCAGGTGATGTGGCCTCAGGACTCCCGTGCTCTGT
CACTCTTGAGACCCAAGCCCTGGCTCCCCAAAGACCTTCTCCGCCCTCCAGCTTTG
CCTTGGTGGAGAAATAAAATCCAAAGCAAGTCagacctcggcttttgtctgtctgtcctccgggccatcactata
gccctcttataaatttctcagtatgatgaccagatgggtgtttgtccctgctcaagtcctgagtaggaacagcctgaccaatgcatcaggttcagcgc
ctactctgcgtagaggggctgcaacctctatgtggtgacataccccaaccaagagagtcacaggtcctgcaagctgccagccacagccaggcct
gggctgggctgcggggcgtcagtcacttaaccgctaatcccttagacaagtctacccgtccatccagggagcctcggaccctgtaggttcttcaa
ggtatggataagaatctctggattaggcaataaagttggaagggcaaaaaggagtcgtttaacagatagagtgggctggagaggctgcctgta
cctctgctcctaccccagccctctgaccagagccctagcatcaaaggcaccaaaaccacagatggccacccaattagtcccccttttcttccaaatt
tccacctgagcagetattcccaagtcctcatctctttccctcctggttcatagtgagcaggtctcaggcccaagcagactacaccaagattcgggtca
geggagagggttgcctctgggaagtcttcctgaagaaaggggatacactatgcctgttctgacacccgagaagtgttaggcagccctcaggcctg
gaggtcacttgggctacctgcccctgactgctgagttcctcacccctcccactggaaccatgagctgacagggtgtgatgtgggagtgcaagtca
atcagtggtctatcacactgggtgtgtcccaggg.

### (2) PCR Amplification

In this example, the reaction system and reaction conditions of PCR amplification were shown in Table 2.

TABLE 2
Reaction system and reaction conditions of PCR amplification

| | PCR system | PCR reaction conditions | | |
|---|---|---|---|---|
| PCR | Template DNA | Pre-denaturation at 98° C. for 2 min | | |
| | (~500 ng/μl):2.5 μl | | | |
| Protocol | Primer S/A (10 μM): each of 1 μl | Denaturation at 98° C. for 20 sec | | |
| | dNTP(2.5 mmol/L): 5 μl | Annealing at 55° C. for 20 sec | {close oversize brace} | 30× cycles |
| | 10x buffer: 5μ1 | Extension at 72° C. for 10 sec | | |
| | Eazy-taq (5 μ/μL)): 0.5 μl | Terminal extension at 72° C. for 5 min | | |
| | Supplementing to | Cooling at 16° C. for 2 min | | |
| | 50 μl with ddH₂O | | | |

(3) The electrophoresis detection was conducted on the amplification product using agarose gel.

### 1) Preparation of 3% Agarose Gel

1.5 g of agarose was placed in a conical flask, added with 50 ml of a 1×TAE buffer (the TAE buffer was composed of Tris base, acetic acid, and EDTA) , and a small beaker was inverted at the bottle mouth. The mixture was boiled by heating in a microwave oven for 3 times until the agarose was completely melted, and shaken well to obtain a 3.0% agarose gel solution.

### 2) Gel Plate Preparation

A plexiglass inner tank (gel preparation tank) in the electrophoresis tank was washed, air-dried, and a gel preparation glass plate was added. The glass plate and edges of both ends of the inner tank were sealed using scotch tape to form a mold. The inner tank was placed in a horizontal position and a comb was placed in a fixed position. The agarose gel liquid cooled to about 65° C. was mixed well and poured on the inner tank glass plate carefully, such that the gel solution was slowly spread until a uniform layer of gel was formed on the entire surface of the glass plate. The glass plate was allowed to stand at room temperature until the gel was completely solidified, the comb was slightly pulled vertically and the scotch tape was removed, then the gel and the inner tank were put into an electrophoresis tank. 1×TAE running buffer was added until the gel plate was immersed.

### 3. Sample Loading

The DNA sample and the loading buffer were mixed on a spot plate or a parafilm, where a final dilution of the loading buffer was not less than lx. The samples were added to the sample grooves of the gel plate by a 10 μl micropipette separately. After adding a sample, the tip for sample loading was replaced to prevent contamination, and the gel surface around the sample well was not damaged when loading the samples. (Note: before loading the samples, a sequence of sample loading was recorded).

### 4) Electrophoresis

After loading the samples, the gel plate was immediately energized for electrophoresis, at a voltage of 60 V to 100 V, and the samples moved from a negative electrode (black) to a positive electrode (red). As the voltage increased, an effective separation range of the agarose gel decreased. When bromophenol blue moved to about 1 cm from a lower edge of the gel plate, the electrophoresis was terminated.

5) After electrophoresis, the gel was removed and stained with a 1×TAE solution containing 0.5 μg/ml ethidium bromide for about 20 min, and then rinsed with water for 10 min.

### 6) Observation and Photography

The gel was observed under ultraviolet light, and the presence of DNA showed a red fluorescent band, which was then photographed and stored by a gel imaging system.

Step 3: identification and discrimination of rats

The heterozygous rats or the homozygous rats were identified according to the electrophoresis result.

Determination basis for PCR amplification results: negative (WT) Ptgds+/+ showed one band: 609 bp (a sequence length between L-S and L-A); heterozygote (HZ) Ptgds+/− showed two bands of 609 bp and 786 bp (the remaining sequence length after knockout of 2,944 bp in wild-type); homozygote (HO) Ptgds−/− showed one band: 786 bp.

In this example, 8 samples (denoted as D33 to D42) were selected, and the Ptgds gene knockout rats were obtained by the above method. The electrophoresis results of PCR amplification were shown in FIG. 2 and FIG. 3. FIG. 2 was L-A&L-S (609 bp); FIG. 3 was R-A/L-S (786 bp).

TABLE 3
Results of phenotype determination of Ptgds knockout rats

| Sample | D33 | D34 | D35 | D36 | D37 | D38 | D39 | D42 |
|---|---|---|---|---|---|---|---|---|
| L-A&L-S | ✓ | ✓ | No | No | No | ✓ | ✓ | ✓ |
| (609 bp) | | | | | | | | |
| R-A&L-S | No | No | ✓ | ✓ | ✓ | ✓ | ✓ | ✓ |
| (786 bp) | | | | | | | | |
| Interpretation | WT | WT | HO | HO | HO | HZ | HZ | HZ |
| of results | | | | | | | | |

According to the electrophoresis results of FIG. 2 and FIG. 3, the determination results in Table 3 were obtained. Of the 8 samples, 2 were negative (WT), 3 were heterozygous (HZ), and 3 were homozygous (HO).

Rats identified as heterozygous (Ptgds+/−) were bred as F0-generation heterozygous rats.

The F0-generation rats and wild-type SD rats were caged together, and the heterozygous rats identified from offspring were used as F1-generation rats; the F1-generation rats were continued to be caged with the wild-type SD rats, and the heterozygous rats identified from offspring were used as F2-generation rats; and rats produced by self-breding within the group of the F2-generation rats were used as F3-generation rats, and homozygous rats (Ptgds−/−) among them were used as the Ptgds gene knockout rat model. In reproduction of each generation, the phenotype of the Ptgds knockout rats in offspring was determined by the above-mentioned genetic identification method.

Breeding passages of F1-F3 lasted 16 months. There were 2 heterozygous rats (2 females) and 8 wild-type rats (3 females, 5 males) produced in the F0 generation; 2 heterozygous rats (1 female, 1 male) and 9 wild-type rats (4 females, 5 males) generation produced in the F1 generation; 5 heterozygous rats (3 females, 2 males) and 5 wild-type rats (2 females, 3 males) produced in the F2 generation; 33 offspring mice, including 4 homozygotes (2 females, 2 males, with a homozygous rate of about 12.12%), 19 heterozygotes (12 females, 7 males), and 9 wild-type mice (5 females, 4 males) bred in the F3 generation. During the breeding, 2 adult mice of F3 generation died, showing a mortality rate of about 6.06%. In the F3 generation, Ptgds wild-type (−/−, 9 rats): heterozygous type (+/−, 19 rats): homozygous type (+/+, 4 rats) had a ratio of approximately 2.25:4.75:1.

### 7. Determination of Kidney Yin Deficiency Indexes in Perimenopause

Perimenopausal kidney yin deficiency is a syndrome manifested by deficiency of kidney yin, lack of nourishment, and internal heat with yin deficiency. The main clinical manifestations are dizziness, tinnitus, insomnia and dreaminess, dysphoria in chestpalms-soles, waist and knee pains, hot flashes, and night sweats. A large number of experimental studies have shown that perimenopausal kidney yin deficiency is related to the dysfunction of the hypothalamic-pituitary-gonadal axis, mainly manifested as abnormal body weight, increased hot flash index, decreased renal function, abnormal blood glucose, lipid metabolism disorders, and disturbance of endocrine hormone levels (estrogen, thyroid hormone, and adrenal cortex hormone) related to the hypothalamic-pituitary-gonadal axis.

Therefore, the related indicators of “hypothalamus-pituitary-gonadal axis” in Ptgds knockout rats with different phenotypes were determined; and the results were subjected to one-way statistical analysis of variance by SPSS software, and p<0.05 indicated that there was a significant statistical difference between the groups.

### (1) Determination of Body Weight and Organ Indexes

In perimenopausal women, due to the rapid depletion of estrogen in the body, may have abnormal weight gain caused by the disorder of fat metabolism. In perimenopausal patients with kidney yin deficiency, may also have typical degenerative changes in their internal organs. Therefore, in this study, body weight and organ index of rats were used as external indicators for the preliminary evaluation of perimenopausal fat metabolism disorder and organ degenerative changes in gene knockout rats.

Since the life span of this type of rats is shorter than that of wild-type rats (12-14 months old), three phenotypes of 8-month-old Ptgds knockout nulliparous female rats (middle-aged, negative, heterozygous and homozygous types) were anesthetized with a 2% sodium pentobarbital solution. The brain, uterus, kidney and spleen of rats were isolated, rinsed with normal saline, dried with filter papers, weighed, and the organ index was calculated (organ index %=organ weight/body weight×100%).

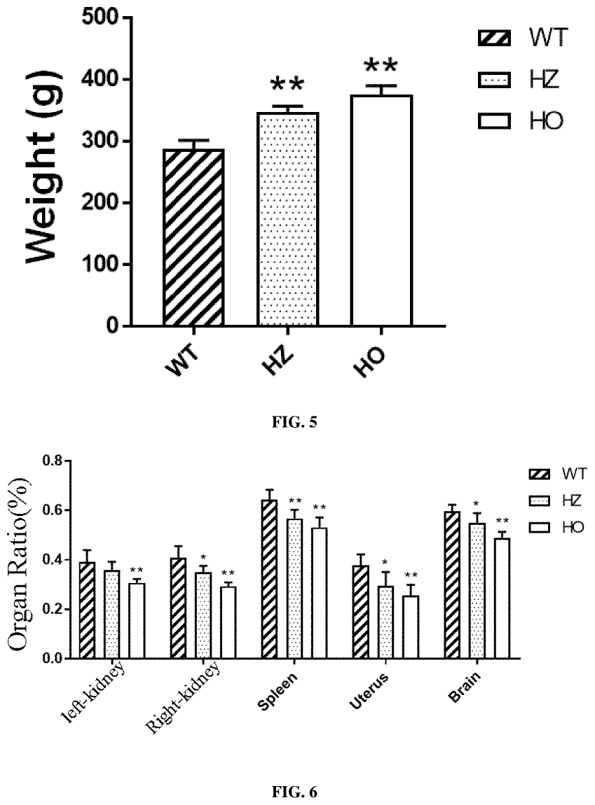

The results were shown in FIG. 5 and FIG. 6. FIG. 5 was a schematic diagram of the body weight of 8 months rats; FIG. 6 was a schematic diagram of the organ index (*p<0.05, **p<0.01, NA: no statistical difference).

Referring to FIG. 5 and FIG. 6, it was seen that homozygous rats had a significant weight gain trend compared with heterozygous and wild-type rats; determining from a change trend of the organ index, the organ indexes of kidney, spleen, uterus and brain of homozygous rats showed obvious degenerative changes.

### (2) Tail Temperature Measurement by Infrared Thermal Imaging

One week after bilateral oophorectomy of rats, a blood flow of the tail increased and showed a transient surge, and a skin temperature of the tail was proportional to the blood flow of the tail, and typical symptoms of perimenopausal hot flashes appeared. Therefore, the severity of hot flashes and the effectiveness of drugs in the treatment of hot flash symptoms were evaluated by measuring the rat tail temperature in real time.

The rat was immobilized in a immobilizer, and a tip of the rat tail was immobilized on a surface of a measuring table; a skin temperature at about 2 cm from the base of the tail of the rat within 6 h was measured with an infrared thermal imager. Data were recorded with a temperature logger, and sampled every 5 min. An average temperature of the first 15 min was used as a baseline value. After the rat was in a stable state in the immobilizer, the temperature value was recorded and a temperature change was evaluated (there were 15 data points, 6 evaluation points, a laboratory temperature was 25° C.±2° C., a measurement time was at 9:00 to 12:00).

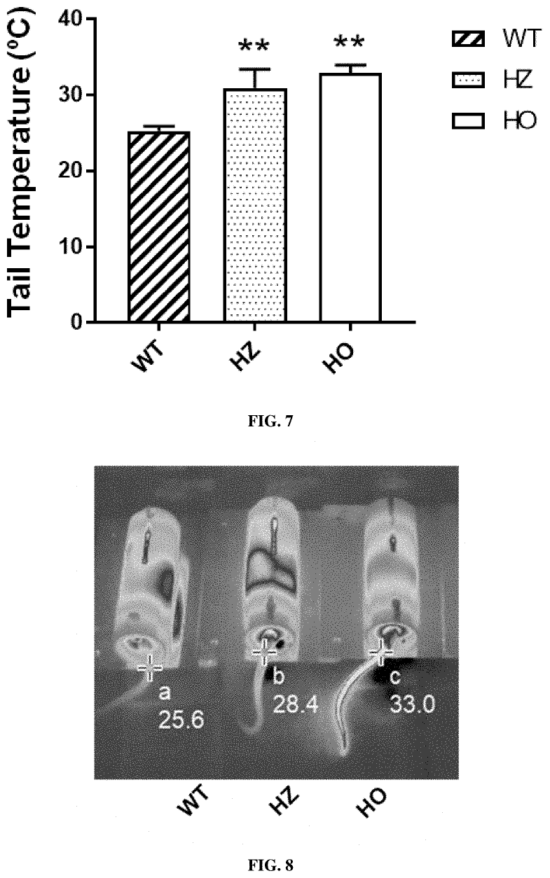

The results were shown in FIG. 7 and FIG. 8. FIG. 7 showed the results of tail temperature measurement; FIG. 8 showed the results of infrared thermal imaging. (*p<0.05, **p<0.01, NA: no statistical difference).

As can be seen from FIG. 7 and FIG. 8, the tail temperatures of the heterozygous and homozygous rats each were significantly higher than that of the wild-type rats (p<0.01); compared with the heterozygous rats, the homozygous rats had more pronounced hot flashes.

### (3) Determination of Function in Kidney, Pancreas, Thyroid and Uterus

Since kidney yin deficiency is related to the hyperfunction of the hypothalamus-pituitary-gonadal axis, the functions of the gonad-related organs (kidney, pancreas, thyroid and uterus) were firstly measured.

Rat serum samples were collected, and blood biochemical indicators were determined enzymatically to evaluate the kidneys, including: albumin (ALB), uric acid (UA), urea (UREA), blood glucose (GLU), total cholesterol (TC), triglyceride (TG), and creatinine (CREA).

The secretion function indexes of kidney, pancreas, thyroid and uterine glands were determined by an enzyme-linked immunosorbent assay (ELISA), including: kidney, uterine ERβ, serum adrenocorticotropic hormone (ACTH), corticosterone (CORT), insulin (INS), cyclic adenosine monophosphate (cAMP), and thyroid stimulating hormone (TSH).

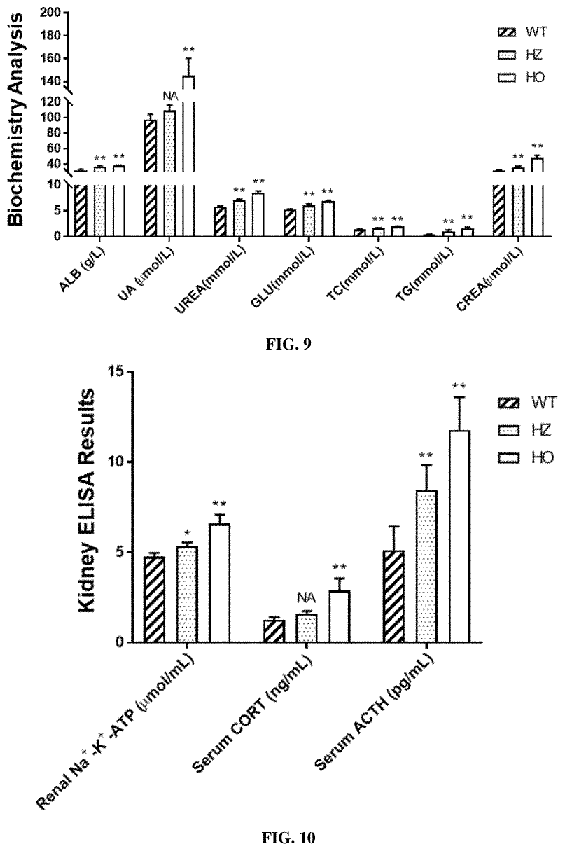

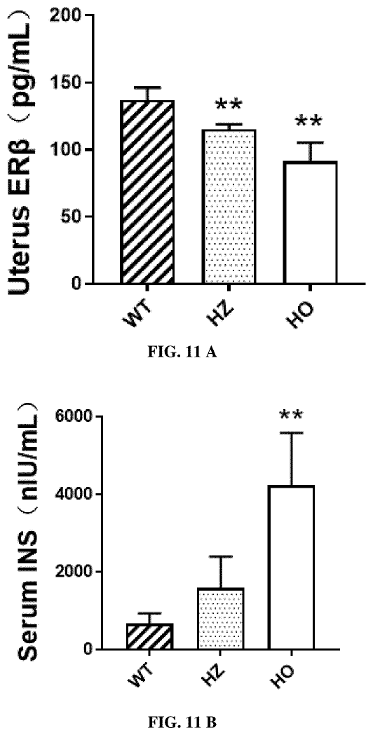

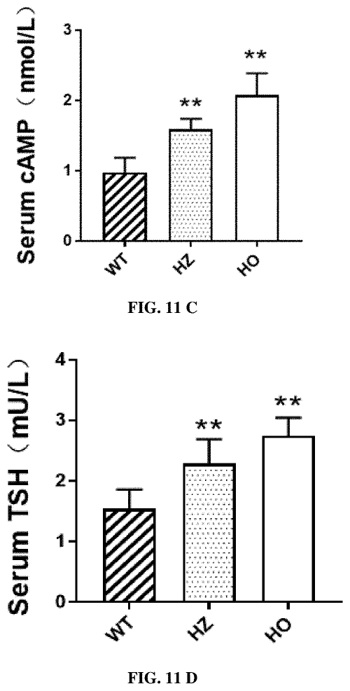

The measurement results were shown in FIG. 9 to FIGS. 11A-D. FIG. 9 showed serum biochemical assay results; FIG. 10 showed the ELISA results of kidney functions; and FIGS. 11A-D showed the ELISA results of other gonad-related organ functions. (*p<0.05, **p<0.01, NA: no statistical difference).

The results of blood biochemical tests showed that the heterozygous and homozygous rats had abnormal renal function and blood lipid indexes compared with the wild-type rats, indicating that the two types of rats might have kidney damage and lipid metabolism disorders. The abnormal increase of renal function Na⁺-K⁺-ATPase, ACTH, CORT, immune cAMP and thyroid TSH further indicated that, the heterozygous and homozygous rats showed typical symptoms of kidney yin deficiency and abnormal renal function and hyperfunction of endocrine glands. Compared with wild-type rats, the decrease of ERβ level in uterus of heterozygous and homozygous rats indicated that the ovarian secretion function of rats was degenerated; this result was consistent with the decreased uterine organ index, indicating that Ptgds knockout rats could simulate the perimenopausal uterine and ovarian decline in rats. The abnormal increase of insulin levels in heterozygous and homozygous rats reflected the abnormal increase of blood glucose and the lipid metabolism disorder caused by insulin resistance in perimenopause.

### (4) Morris Water Maze Test

Perimenopause patients with kidney yin deficiency may show typical symptoms of central degenerative diseases such as lapse of memory and senile dementia. Therefore, the memory and learning ability of Ptgds knockout rats were evaluated by a Morris water maze method. Experiments were conducted in a Morris water maze system, including a white plastic pool (130 cm in diameter, 50 cm in height, with a built-in PVC cylindrical escape platform), an automatic camera, and image tracking processing software.

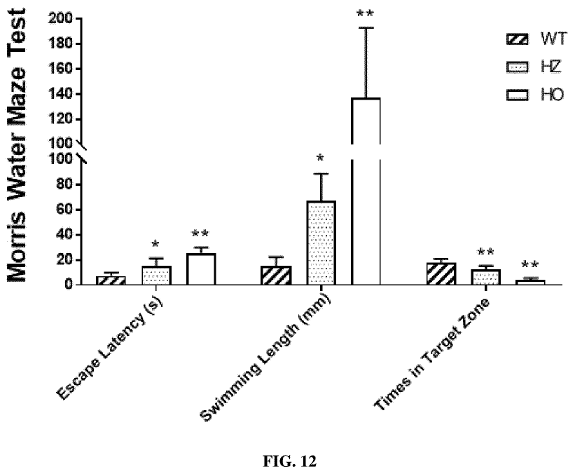

Before the test, a 5-day positioning navigation training was conducted. That is, each rat (stained with yellow dye on a top of the head) was placed in titanium powder-dyed white water (23° C.±2° C.) with its head facing the pool wall at random quadrant points, and timing started at the moment of release; the timing was stopped when the rat touched the escape platform and stayed there for at least 3 sec. When finding the platform, the rats stayed on the platform for 10 sec; if the rat could not find the platform within 90 sec, the rats were guided to the platform and acclimated for 10 sec. After the formal experiment began, the original escape platform was removed; the second quadrant was used as a fixed water entry quadrant, and the escape latency, swimming distance, and times of crossing the platform of the rats were recorded within a specified time, to evaluate the learning and memory ability of the rats. The results were shown in FIG. 12.

Referring to FIG. 12, it was seen that compared with wild-type rats, the escape latency and swimming distance of heterozygous and homozygous rats increased significantly, while the times of crossing the platform was significantly reduced, especially in homozygous rats (p<0.01). This indicated that the level of learning and memory ability of heterozygous and homozygous rats was significantly lower than that of normal rats.

In summary, the present disclosure is based on use of the CRISPR/Cas9 technology in targeted knockout of Ptgds gene in rats, thereby constructing a Ptgds −/− rat model to produce symptoms of the spontaneous kidney yin deficiency. Through breeding, PCR identification, and pathological index detection, the model can be further applied to pharmacodynamic evaluation. The model can provide a reliable and stable genetic engineering model for the study of perimenopausal symptoms of kidney yin deficiency, and lay a foundation for exploration of perimenopausal syndrome mechanism and evaluation of related drug treatment effects.

The foregoing are merely descriptions of the specific embodiments of the present disclosure, and the claimed scope of the present disclosure is not limited thereto. Any modification, equivalent replacement, improvement, etc. made within the technical scope of the present disclosure by those skilled in the art according to the spirit and principle of the present disclosure shall fall within the claimed scope of the present disclosure.

| | |
|---|---|
| Sequence Listing Information: | |
| DTD Version: V1_3 | |
| File Name: GWP20220801139.xml | |
| Software Name: WIPO Sequence | |
| Software Version: 2.1.2 | |
| Production Date: 2022-12-13 | |
| General Information: | |
| Current application / Applicant file reference: GWP20220801139 | |
| Earliest priority application / IP Office: CN | |
| Earliest priority application / Application number: 202210191036.7 | |
| Earliest priority application / Filing date: 2022-02-25 | |
| Applicant name: Shaanxi University of Chinese Medicine | |
| Applicant name / Language: en | |
| Invention title: METHOD FOR CONSTRUCTING Ptgds GENE KNOCKOUT RAT MODEL | |
| WITH SPONTANEOUS KIDNEY YIN DEFICIENCY ( en ) | |
| Sequence Total Quantity: 13 | |
| Sequences: | |
| Sequence Number (ID): 1 | |
| Length: 23 | |
| Molecule Type: DNA | |
| Features Location/Qualifiers: | |
| - source, 1..23 | |
| > mol_type, other DNA | |
| > note, Ptgds-sgRNA1 | |
| > organism, synthetic construct | |
| Residues: | |
| ccacgttgct ggcctcaggc tca | 23 |
| Sequence Number (ID): 2 | |
| Length: 23 | |
| Molecule Type: DNA | |
| Features Location/Qualifiers: | |
| - source, 1..23 | |
| > mol_type, other DNA | |
| > note, Reverse complement of Ptgds-sgRNA1 | |
| > organism, synthetic construct | |
| Residues: | |
| tgagcctgag gccagcaacg tgg | 23 |
| Sequence Number (ID): 3 | |
| Length: 23 | |
| Molecule Type: DNA | |
| Features Location/Qualifiers: | |
| - source, 1..23 | |
| > mol_type, other DNA | |
| > note, Ptgds-sgRNA2 | |
| > organism, synthetic construct | |
| Residues: | |
| ccaaagcaag tcagacctcg gct | 23 |
| Sequence Number (ID): 4 | |
| Length: 23 | |
| Molecule Type: DNA | |
| Features Location/Qualifiers: | |
| - source, 1..23 | |
| > mol_type, other DNA | |
| > note, Reverse complement of Ptgds-sgRNA2 | |
| > organism, synthetic construct | |
| Residues: | |
| agccgaggtc tgacttgctt tgg | 23 |
| Sequence Number (ID): 5 | |
| Length: 15 | |
| Molecule Type: DNA | |
| Features Location/Qualifiers: | |
| - source, 1..15 | |
| > mol_type, other DNA | |
| > note, Nucleotide sequence of the intron sequence fragment | |
| > organism, synthetic construct | |
| Residues: | |
| tgcccacgtt gctgg | 15 |
| Sequence Number (ID): 6 | |
| Length: 2935 | |
| Molecule Type: DNA | |
| Features Location/Qualifiers: | |
| - source, 1..2935 | |
| > mol_type, other DNA | |
| > note, Nucleotide sequence of the exon sequence fragment | |
| > organism, synthetic construct | |
| Residues: | |
| cctcaggctc agacacctgc tctactccaa gcaaatggct gctcttccaa tgctgtggac | 60 |
| cgggctggtc ctcttgggtc tcttgggatt tccacagacc ccagcccagg gccatgacac | 120 |
| agtgcagccc aactttcaac aagacaaggt gagagggtcc cctaccccac acccgaggaa | 180 |
| acagaaacct caggtcagag ccaggctttc tctcacaaga gagggtgcgt tgggcgctgt | 240 |
| cagccatggg agctgtctgg aaccgcgctg gcacacagcc tggttggtcc acctgactcc | 300 |
| gccaggaatg tggctctgat acccacttta ccggaagagt agactggggc gagcactggg | 360 |
| acaaagacgg gagctcaaca tcctggggaa ggaaggggtc aatgaggcaa tgagccagcc | 420 |
| tactagagag agagaggggc gtggatgcta ccagaacctg tgtgtgggag gagtcagagt | 480 |
| agggaaggcc agcccactag ggtctgccca tgaggggcgc atggtgcaga cccgggcatc | 540 |
| cactggtcac agttcctggg gcgctggtac agcgcgggcc tcgcctccaa ttcaagctgg | 600 |
| ttccgggaga agaaagagct actgtttatg tgccagacag tggtagctcc ctccacagaa | 660 |
| ggcggcctca acctcacctc taccttccta aggtgagaca agggggtgtg gcaagtttcg | 720 |
| ggacagaagg ccccacaacc ctgtctgggg gacatcctgg ggcttgttcc cttacatcag | 780 |
| gggtaatcta cccacaggaa aaaccagtgt gagaccaagg tgatggtact gcagccggca | 840 |
| ggggttcccg gacagtacac ctacaacagc ccccgtgagt gagccacttc cttatctggg | 900 |
| taaattctga ggtaaatgct ggcagactgt gcagccccct gtcccaaaag gtggggataa | 960 |
| tggtcacacc acaagggtca gtcatccaag accagacctg attgtgaatc tgcctcaggc | 1020 |
| acacagggct acctctctcc agggactttg gcctctctga aacccagcca cattcttcca | 1080 |
| ggcccctttc ctgtccaaat gaaatttccc agtactctgc tgcccaagtg ggtcacatac | 1140 |
| aggcattccc caaatcctac ccacatttca tagctcctat ccaagtacct ctttccatgc | 1200 |
| ctcacctgat ctatggattc ccaccagaac cctatttcct tggccttcct gctatattgt | 1260 |
| aactcagcct gatgatttct tgagtctaag tgttttctgc cctctcccca agattcatgg | 1320 |
| tttggagtta gtgttcagga aggaagctag agattgggtg gtggccaccc aggggagcac | 1380 |
| agggaaagaa gccaaagcag gggtggagga ggaaggcctg agaccctccc cacagagaag | 1440 |
| cccacaaagg ccaccccctc caagcagagg gagatagtga tgtgggagcc acatgtctta | 1500 |
| atcagtgtca tttcttgggt tcccagactg gggcagcttc cactccctct cagtggtaga | 1560 |
| aaccgactac gatgagtacg cgttcctgtt cagcaagggc accaagggcc caggccagga | 1620 |
| cttccgcatg gccaccctct acagtaggta tcccagccca caggcccacg cacagggcag | 1680 |
| atgcctgagg ttggaaacag accaaggcct aacccagagg acagtaacga aggtgtgtgg | 1740 |
| gggcagggcg agggcttttc acctcctgac accggcccct tctttatcta ccaggcagag | 1800 |
| cccagcttct gaaggaggaa ctgaaggaga aattcatcac ctttagcaag gaccagggcc | 1860 |
| tcacagagga ggacattgtt ttcctgcccc aaccgggtga gggaggctaa gctgctgagg | 1920 |
| agggaattag tgcagattag tgcagcctgt ggactgggga gagtgtggcc gcctactagt | 1980 |
| ccaggggctc caaggaaaga aatggaggtg tcagtctgtc ccgacagtac ctcgacctgc | 2040 |
| agcccccttt attgggaacc ctcttcctgg tggacacctc gctgccctgt ctgccagccc | 2100 |
| cctagctagg gatttagggg cactaacaga tggagaaaga caccttttat gttttaaaga | 2160 |
| acagattgga gcaggagtgg gatggagtct gaagtgtggg gctcagcctt ggggaggctt | 2220 |
| cgtaaagtcc agggagaaga caaagtcctg gtgactgtgg gtctaagcct gatactgact | 2280 |
| acttccctgg gcttctttct caacagataa gtgcattcaa gagtaaacac aggtgagaga | 2340 |
| agtcagtcac aggtaacaca tggtaagtgc catttactca ctcaacataa gaccactgag | 2400 |
| tgctcatgtg accacggagt gcgggctggg gtggggggga tgcagctgcc caaggactgt | 2460 |
| ccaagtgaga cagccagaga gaaaggacag ttccaattcc agtggcagga atagagctga | 2520 |
| tggccaaggg ttcatgggag aaggataaca gcaatgggaa gggaccgccc catgaagccc | 2580 |
| atcctgcaaa atgagtctcc aaggaaccag aatggacaag atcgggaagg gactggtggc | 2640 |
| cagggatgga catggcgagt cagagggctg gctcctcacc tgtgctgttg actgagactc | 2700 |
| tgagaccata ggccctggag ggatacccta ggaggccctg gccggaagtg ttgtttgggc | 2760 |
| cccactgggc tcagggtgct gccctcatca ctgatggctc ttgttcttct gtgcaggtga | 2820 |
| tgtggcctca ggactcccgt gctctgtcac tcttgagacc caagccctgg ctccccaaag | 2880 |
| accttctccg ccctccagct ttgccttggt ggagaaataa aatccaaagc aagtc | 2935 |
| Sequence Number (ID): 7 | |
| Length: 20 | |
| Molecule Type: DNA | |
| Features Location/Qualifiers: | |
| - source, 1..20 | |
| > mol_type, other DNA | |
| > note, Primer Ptgds-L-S | |
| > organism, synthetic construct | |
| Residues: | |
| gcaggtctta gccataggtg | 20 |
| Sequence Number (ID): 8 | |
| Length: 18 | |
| Molecule Type: DNA | |
| Features Location/Qualifiers: | |
| - source, 1..18 | |
| > mol_type, other DNA | |
| > note, Primer Ptgds-L-A | |
| > organism, synthetic construct | |
| Residues: | |
| tttctgtttc ctcgggtg | 18 |
| Sequence Number (ID): 9 | |
| Length: 21 | |
| Molecule Type: DNA | |
| Features Location/Qualifiers: | |
| - source, 1..21 | |
| > mol_type, other DNA | |
| > note, Primer Ptgds-R-S | |
| > organism, synthetic construct | |
| Residues: | |
| gatggctctt gttcttctgt g | 21 |
| Sequence Number (ID): 10 | |
| Length: 22 | |
| Molecule Type: DNA | |
| Features Location/Qualifiers: | |
| - source, 1..22 | |
| > mol_type, other DNA | |
| > note, Primer Ptgds-R-A | |
| > organism, synthetic construct | |
| Residues: | |
| ccttccaact ttattgccta at | 22 |
| Sequence Number (ID): 11 | |
| Length: 4299 | |
| Molecule Type: DNA | |
| Features Location/Qualifiers: | |
| - source, 1..4299 | |
| > mol_type, other DNA | |
| > organism, synthetic construct | |
| Residues: | |
| tagcctttca ggaccaaatg ttcaaggcac agatggttct ttgtgttccc tgctggggtc | 60 |
| atgggacttg gaaagggatg gtaggtaggg cttgtgagaa gcaggtctta gccataggtg | 120 |
| ggcagtgact agattttcca gcagctggga agctccagag tacacatccg gcaccatgtg | 180 |
| aggtatgtgg gctttgctgg cagggtggac aaggtctgag ccacttctgc ctctggagtt | 240 |
| ggggaggggg gacaggcaga ggcctctgcc tgccctgccc tgctgacctg cccctgcccg | 300 |
| ttcttcactg aggtatgggg ctctgctgga gcctcttaca taatgaacag atgaggctgc | 360 |
| agctggggca gccgcccgcc ctccctcaca ccagcatcac gagcctccag tgggcagtcc | 420 |
| ttgggccttg ggtggaggcc aagcctggtt cataaatagg gtctccacgg tggcctctgc | 480 |
| tccatctgcc cacagtcttc cttgctttgc ccacgttgct ggcctcaggc tcagacacct | 540 |
| gctctactcc aagcaaatgg ctgctcttcc aatgctgtgg accgggctgg tcctcttggg | 600 |
| tctcttggga tttccacaga ccccagccca gggccatgac acagtgcagc ccaactttca | 660 |
| acaagacaag gtgagagggt cccctacccc acacccgagg aaacagaaac ctcaggtcag | 720 |
| agccaggctt tctctcacaa gagagggtgc gttgggcgct gtcagccatg ggagctgtct | 780 |
| ggaaccgcgc tggcacacag cctggttggt ccacctgact ccgccaggaa tgtggctctg | 840 |
| atacccactt taccggaaga gtagactggg gcgagcactg ggacaaagac gggagctcaa | 900 |
| catcctgggg aaggaagggg tcaatgaggc aatgagccag cctactagag agagagaggg | 960 |
| gcgtggatgc taccagaacc tgtgtgtggg aggagtcaga gtagggaagg ccagcccact | 1020 |
| agggtctgcc catgaggggc gcatggtgca gacccgggca tccactggtc acagttcctg | 1080 |
| gggcgctggt acagcgcggg cctcgcctcc aattcaagct ggttccggga gaagaaagag | 1140 |
| ctactgttta tgtgccagac agtggtagct ccctccacag aaggcggcct caacctcacc | 1200 |
| tctaccttcc taaggtgaga caagggggtg tggcaagttt cgggacagaa ggccccacaa | 1260 |
| ccctgtctgg gggacatcct ggggcttgtt cccttacatc aggggtaatc tacccacagg | 1320 |
| aaaaaccagt gtgagaccaa ggtgatggta ctgcagccgg caggggttcc cggacagtac | 1380 |
| acctacaaca gcccccgtga gtgagccact tccttatctg ggtaaattct gaggtaaatg | 1440 |
| ctggcagact gtgcagcccc ctgtcccaaa aggtggggat aatggtcaca ccacaagggt | 1500 |
| cagtcatcca agaccagacc tgattgtgaa tctgcctcag gcacacaggg ctacctctct | 1560 |
| ccagggactt tggcctctct gaaacccagc cacattcttc caggcccctt tcctgtccaa | 1620 |
| atgaaatttc ccagtactct gctgcccaag tgggtcacat acaggcattc cccaaatcct | 1680 |
| acccacattt catagctcct atccaagtac ctctttccat gcctcacctg atctatggat | 1740 |
| tcccaccaga accctatttc cttggccttc ctgctatatt gtaactcagc ctgatgattt | 1800 |
| cttgagtcta agtgttttct gccctctccc caagattcat ggtttggagt tagtgttcag | 1860 |
| gaaggaagct agagattggg tggtggccac ccaggggagc acagggaaag aagccaaagc | 1920 |
| aggggtggag gaggaaggcc tgagaccctc cccacagaga agcccacaaa ggccaccccc | 1980 |
| tccaagcaga gggagatagt gatgtgggag ccacatgtct taatcagtgt catttcttgg | 2040 |
| gttcccagac tggggcagct tccactccct ctcagtggta gaaaccgact acgatgagta | 2100 |
| cgcgttcctg ttcagcaagg gcaccaaggg cccaggccag gacttccgca tggccaccct | 2160 |
| ctacagtagg tatcccagcc cacaggccca cgcacagggc agatgcctga ggttggaaac | 2220 |
| agaccaaggc ctaacccaga ggacagtaac gaaggtgtgt gggggcaggg cgagggcttt | 2280 |
| tcacctcctg acaccggccc cttctttatc taccaggcag agcccagctt ctgaaggagg | 2340 |
| aactgaagga gaaattcatc acctttagca aggaccaggg cctcacagag gaggacattg | 2400 |
| ttttcctgcc ccaaccgggt gagggaggct aagctgctga ggagggaatt agtgcagatt | 2460 |
| agtgcagcct gtggactggg gagagtgtgg ccgcctacta gtccaggggc tccaaggaaa | 2520 |
| gaaatggagg tgtcagtctg tcccgacagt acctcgacct gcagccccct ttattgggaa | 2580 |
| ccctcttcct ggtggacacc tcgctgccct gtctgccagc cccctagcta gggatttagg | 2640 |
| ggcactaaca gatggagaaa gacacctttt atgttttaaa gaacagattg gagcaggagt | 2700 |
| gggatggagt ctgaagtgtg gggctcagcc ttggggagge ttcgtaaagt ccagggagaa | 2760 |
| gacaaagtcc tggtgactgt gggtctaagc ctgatactga ctacttccct gggcttcttt | 2820 |
| ctcaacagat aagtgcattc aagagtaaac acaggtgaga gaagtcagtc acaggtaaca | 2880 |
| catggtaagt gccatttact cactcaacat aagaccactg agtgctcatg tgaccacgga | 2940 |
| gtgcgggctg gggtgggggg gatgcagctg cccaaggact gtccaagtga gacagccaga | 3000 |
| gagaaaggac agttccaatt ccagtggcag gaatagagct gatggccaag ggttcatggg | 3060 |
| agaaggataa cagcaatggg aagggaccgc cccatgaagc ccatcctgca aaatgagtct | 3120 |
| ccaaggaacc agaatggaca agatcgggaa gggactggtg gccagggatg gacatggcga | 3180 |
| gtcagagggc tggctcctca cctgtgctgt tgactgagac tctgagacca taggccctgg | 3240 |
| agggataccc taggaggccc tggccggaag tgttgtttgg gccccactgg gctcagggtg | 3300 |
| ctgccctcat cactgatggc tcttgttctt ctgtgcaggt gatgtggcct caggactccc | 3360 |
| gtgctctgtc actcttgaga cccaagccct ggctccccaa agaccttctc cgccctccag | 3420 |
| ctttgccttg gtggagaaat aaaatccaaa gcaagtcaga cctcggcttt tgtctgtctg | 3480 |
| tcctccgggc catcactata gccctcttat aaatttctca gtatgatgac cagatgggtg | 3540 |
| tttgtccctg ctcaagtcct gagtaggaac agcctgacca atgcatcagg ttcagcgcct | 3600 |
| actctgcgta gaggggctgc aacctctatg tggtgacata ccccaaccaa gagagtcaca | 3660 |
| ggtcctgcaa gctgccagcc acagccaggc ctgggctggg ctgcggggcg tcagtcactt | 3720 |
| aaccgctaat cccttagaca agtctacccg tccatccagg gagcctcgga ccctgtaggt | 3780 |
| tcttcaaggt atggataaga atctctggat taggcaataa agttggaagg gcaaaaagga | 3840 |
| gtcgtttaac agatagagtg ggctggagag gctgcctgta cctctgctcc taccccagcc | 3900 |
| ctctgaccag agccctagca tcaaaggcac caaaaccaca gatggccacc caattagtcc | 3960 |
| cccttttctt ccaaatttcc acctgagcag ctattcccaa gtcctcatct ctttccctcc | 4020 |
| tggttcatag tgagcaggtc tcaggcccaa gcagactaca ccaagattcg ggtcagcgga | 4080 |
| gagggttgcc tctgggaagt cttcctgaag aaaggggata cactatgcct gttctgacac | 4140 |
| ccgagaagtg ttaggcagcc ctcaggcctg gaggtcactt gggctacctg cccctgactg | 4200 |
| ctgagttcct cacccctccc actggaacca tgagctgaca gggtgtgatg tgggagtgca | 4260 |
| agtcaatcag tggtctatca cactgggtgt gtcccaggg | 4299 |
| Sequence Number (ID): 12 | |
| Length: 4299 | |
| Molecule Type: DNA | |
| Features Location/Qualifiers: | |
| - source, 1..4299 | |
| > mol_type, other DNA | |
| > organism, synthetic construct | |
| Residues: | |
| tagcctttca ggaccaaatg ttcaaggcac agatggttct ttgtgttccc tgctggggtc | 60 |
| atgggacttg gaaagggatg gtaggtaggg cttgtgagaa gcaggtctta gccataggtg | 120 |
| ggcagtgact agattttcca gcagctggga agctccagag tacacatccg gcaccatgtg | 180 |
| aggtatgtgg gctttgctgg cagggtggac aaggtctgag ccacttctgc ctctggagtt | 240 |
| ggggaggggg gacaggcaga ggcctctgcc tgccctgccc tgctgacctg cccctgcccg | 300 |
| ttcttcactg aggtatgggg ctctgctgga gcctcttaca taatgaacag atgaggctgc | 360 |
| agctggggca gccgcccgcc ctccctcaca ccagcatcac gagcctccag tgggcagtcc | 420 |
| ttgggccttg ggtggaggcc aagcctggtt cataaatagg gtctccacgg tggcctctgc | 480 |
| tccatctgcc cacagtcttc cttgctttgc ccacgttgct ggcctcaggc tcagacacct | 540 |
| gctctactcc aagcaaatgg ctgctcttcc aatgctgtgg accgggctgg tcctcttggg | 600 |
| tctcttggga tttccacaga ccccagccca gggccatgac acagtgcagc ccaactttca | 660 |
| acaagacaag gtgagagggt cccctacccc acacccgagg aaacagaaac ctcaggtcag | 720 |
| agccaggctt tctctcacaa gagagggtgc gttgggcgct gtcagccatg ggagctgtct | 780 |
| ggaaccgcgc tggcacacag cctggttggt ccacctgact ccgccaggaa tgtggctctg | 840 |
| atacccactt taccggaaga gtagactggg gcgagcactg ggacaaagac gggagctcaa | 900 |
| catcctgggg aaggaagggg tcaatgaggc aatgagccag cctactagag agagagaggg | 960 |
| gcgtggatgc taccagaacc tgtgtgtggg aggagtcaga gtagggaagg ccagcccact | 1020 |
| agggtctgcc catgaggggc gcatggtgca gacccgggca tccactggtc acagttcctg | 1080 |
| gggcgctggt acagcgcggg cctcgcctcc aattcaagct ggttccggga gaagaaagag | 1140 |
| ctactgttta tgtgccagac agtggtagct ccctccacag aaggcggcct caacctcacc | 1200 |
| tctaccttcc taaggtgaga caagggggtg tggcaagttt cgggacagaa ggccccacaa | 1260 |
| ccctgtctgg gggacatcct ggggcttgtt cccttacatc aggggtaatc tacccacagg | 1320 |
| aaaaaccagt gtgagaccaa ggtgatggta ctgcagccgg caggggttcc cggacagtac | 1380 |
| acctacaaca gcccccgtga gtgagccact tccttatctg ggtaaattct gaggtaaatg | 1440 |
| ctggcagact gtgcagcccc ctgtcccaaa aggtggggat aatggtcaca ccacaagggt | 1500 |
| cagtcatcca agaccagacc tgattgtgaa tctgcctcag gcacacaggg ctacctctct | 1560 |
| ccagggactt tggcctctct gaaacccagc cacattcttc caggcccctt tcctgtccaa | 1620 |
| atgaaatttc ccagtactct gctgcccaag tgggtcacat acaggcattc cccaaatcct | 1680 |
| acccacattt catagctcct atccaagtac ctctttccat gcctcacctg atctatggat | 1740 |
| tcccaccaga accctatttc cttggccttc ctgctatatt gtaactcagc ctgatgattt | 1800 |
| cttgagtcta agtgttttct gccctctccc caagattcat ggtttggagt tagtgttcag | 1860 |
| gaaggaagct agagattggg tggtggccac ccaggggagc acagggaaag aagccaaagc | 1920 |
| aggggtggag gaggaaggcc tgagaccctc cccacagaga agcccacaaa ggccaccccc | 1980 |
| tccaagcaga gggagatagt gatgtgggag ccacatgtct taatcagtgt catttcttgg | 2040 |
| gttcccagac tggggcagct tccactccct ctcagtggta gaaaccgact acgatgagta | 2100 |
| cgcgttcctg ttcagcaagg gcaccaaggg cccaggccag gacttccgca tggccaccct | 2160 |
| ctacagtagg tatcccagcc cacaggccca cgcacagggc agatgcctga ggttggaaac | 2220 |
| agaccaaggc ctaacccaga ggacagtaac gaaggtgtgt gggggcaggg cgagggcttt | 2280 |
| tcacctcctg acaccggccc cttctttatc taccaggcag agcccagctt ctgaaggagg | 2340 |
| aactgaagga gaaattcatc acctttagca aggaccaggg cctcacagag gaggacattg | 2400 |
| ttttcctgcc ccaaccgggt gagggaggct aagctgctga ggagggaatt agtgcagatt | 2460 |
| agtgcagcct gtggactggg gagagtgtgg ccgcctacta gtccaggggc tccaaggaaa | 2520 |
| gaaatggagg tgtcagtctg tcccgacagt acctcgacct gcagccccct ttattgggaa | 2580 |
| ccctcttcct ggtggacacc tcgctgccct gtctgccagc cccctagcta gggatttagg | 2640 |
| ggcactaaca gatggagaaa gacacctttt atgttttaaa gaacagattg gagcaggagt | 2700 |
| gggatggagt ctgaagtgtg gggctcagcc ttggggaggc ttcgtaaagt ccagggagaa | 2760 |
| gacaaagtcc tggtgactgt gggtctaagc ctgatactga ctacttccct gggcttcttt | 2820 |
| ctcaacagat aagtgcattc aagagtaaac acaggtgaga gaagtcagtc acaggtaaca | 2880 |
| catggtaagt gccatttact cactcaacat aagaccactg agtgctcatg tgaccacgga | 2940 |
| gtgcgggctg gggtgggggg gatgcagctg cccaaggact gtccaagtga gacagccaga | 3000 |
| gagaaaggac agttccaatt ccagtggcag gaatagagct gatggccaag ggttcatggg | 3060 |
| agaaggataa cagcaatggg aagggaccgc cccatgaagc ccatcctgca aaatgagtct | 3120 |
| ccaaggaacc agaatggaca agatcgggaa gggactggtg gccagggatg gacatggcga | 3180 |
| gtcagagggc tggctcctca cctgtgctgt tgactgagac tctgagacca taggccctgg | 3240 |
| agggataccc taggaggccc tggccggaag tgttgtttgg gccccactgg gctcagggtg | 3300 |
| ctgccctcat cactgatggc tcttgttctt ctgtgcaggt gatgtggcct caggactccc | 3360 |
| gtgctctgtc actcttgaga cccaagccct ggctccccaa agaccttctc cgccctccag | 3420 |
| ctttgccttg gtggagaaat aaaatccaaa gcaagtcaga cctcggcttt tgtctgtctg | 3480 |
| tcctccgggc catcactata gccctcttat aaatttctca gtatgatgac cagatgggtg | 3540 |
| tttgtccctg ctcaagtcct gagtaggaac agcctgacca atgcatcagg ttcagcgcct | 3600 |
| actctgcgta gaggggctgc aacctctatg tggtgacata ccccaaccaa gagagtcaca | 3660 |
| ggtcctgcaa gctgccagcc acagccaggc ctgggctggg ctgcggggcg tcagtcactt | 3720 |
| aaccgctaat cccttagaca agtctacccg tccatccagg gagcctcgga ccctgtaggt | 3780 |
| tcttcaaggt atggataaga atctctggat taggcaataa agttggaagg gcaaaaagga | 3840 |
| gtcgtttaac agatagagtg ggctggagag gctgcctgta cctctgctcc taccccagcc | 3900 |
| ctctgaccag agccctagca tcaaaggcac caaaaccaca gatggccacc caattagtcc | 3960 |
| cccttttctt ccaaatttcc acctgagcag ctattcccaa gtcctcatct ctttccctcc | 4020 |
| tggttcatag tgagcaggtc tcaggcccaa gcagactaca ccaagattcg ggtcagcgga | 4080 |
| gagggttgcc tctgggaagt cttcctgaag aaaggggata cactatgcct gttctgacac | 4140 |
| ccgagaagtg ttaggcagcc ctcaggcctg gaggtcactt gggctacctg cccctgactg | 4200 |
| ctgagttcct cacccctccc actggaacca tgagctgaca gggtgtgatg tgggagtgca | 4260 |
| agtcaatcag tggtctatca cactgggtgt gtcccaggg | 4299 |
| Sequence Number (ID): 13 | |
| Length: 2929 | |
| Molecule Type: DNA | |
| Features Location/Qualifiers: | |
| - source, 1..2929 | |
| > mol_type, other DNA | |
| > note, A sequence fragment between exon 1 and exon 7 of Ptgds gene | |
| > organism, synthetic construct | |
| Residues: | |
| cctcaggctc agacacctgc tctactccaa gcaaatggct gctcttccaa tgctgtggac | 60 |
| cgggctggtc ctcttgggtc tcttgggatt tccacagacc ccagcccagg gccatgacac | 120 |
| agtgcagccc aactttcaac aagacaaggt gagagggtcc cctaccccac acccgaggaa | 180 |
| acagaaacct caggtcagag ccaggctttc tctcacaaga gagggtgcgt tgggcgctgt | 240 |
| cagccatggg agctgtctgg aaccgcgctg gcacacagcc tggttggtcc acctgactcc | 300 |
| gccaggaatg tggctctgat acccacttta ccggaagagt agactggggc gagcactggg | 360 |
| acaaagacgg gagctcaaca tcctggggaa ggaaggggtc aatgaggcaa tgagccagcc | 420 |
| tactagagag agagaggggc gtggatgcta ccagaacctg tgtgtgggag gagtcagagt | 480 |
| agggaaggcc agcccactag ggtctgccca tgaggggcgc atggtgcaga cccgggcatc | 540 |
| cactggtcac agttcctggg gcgctggtac agcgcgggcc tcgcctccaa ttcaagctgg | 600 |
| ttccgggaga agaaagagct actgtttatg toccagacag tggtagctcc ctccacagaa | 660 |
| ggcggcctca acctcacctc taccttccta aggtgagaca agggggtgtg gcaagtttcg | 720 |
| ggacagaagg ccccacaacc ctgtctgggg gacatcctgg ggcttgttcc cttacatcag | 780 |
| gggtaatcta cccacaggaa aaaccagtgt gagaccaagg tgatggtact gcagccggca | 840 |
| ggggttcccg gacagtacac ctacaacagc ccccgtgagt gagccacttc cttatctggg | 900 |
| taaattctga ggtaaatgct ggcagactgt gcagccccct gtcccaaaag gtggggataa | 960 |
| tggtcacacc acaagggtca gtcatccaag accagacctg attgtgaatc tgcctcaggc | 1020 |
| acacagggct acctctctcc agggactttg gcctctctga aacccagcca cattcttcca | 1080 |
| ggcccctttc ctgtccaaat gaaatttccc agtactctgc tgcccaagtg ggtcacatac | 1140 |
| aggcattccc caaatcctac ccacatttca tagctcctat ccaagtacct ctttccatgc | 1200 |
| ctcacctgat ctatggattc ccaccagaac cctatttcct tggccttcct gctatattgt | 1260 |
| aactcagcct gatgatttct tgagtctaag tgttttctgc cctctcccca agattcatgg | 1320 |
| tttggagtta gtgttcagga aggaagctag agattgggtg gtggccaccc aggggagcac | 1380 |
| agggaaagaa gccaaagcag gggtggagga ggaaggcctg agaccctccc cacagagaag | 1440 |
| cccacaaagg ccaccccctc caagcagagg gagatagtga tgtgggagcc acatgtctta | 1500 |
| atcagtgtca tttcttgggt tcccagactg gggcagcttc cactccctct cagtggtaga | 1560 |
| aaccgactac gatgagtacg cgttcctgtt cagcaagggc accaagggcc caggccagga | 1620 |
| cttccgcatg gccaccctct acagtaggta tcccagccca caggcccacg cacagggcag | 1680 |
| atgcctgagg ttggaaacag accaaggcct aacccagagg acagtaacga aggtgtgtgg | 1740 |
| gggcagggcg agggcttttc acctcctgac accggcccct tctttatcta ccaggcagag | 1800 |
| cccagcttct gaaggaggaa ctgaaggaga aattcatcac ctttagcaag gaccagggcc | 1860 |
| tcacagagga ggacattgtt ttcctgcccc aaccgggtga gggaggctaa gctgctgagg | 1920 |
| agggaattag tgcagattag tgcagcctgt ggactgggga gagtgtggcc gcctactagt | 1980 |
| ccaggggctc caaggaaaga aatggaggtg tcagtctgtc ccgacagtac ctcgacctgc | 2040 |
| agcccccttt attgggaacc ctcttcctgg tggacacctc gctgccctgt ctgccagccc | 2100 |
| cctagctagg gatttagggg cactaacaga tggagaaaga caccttttat gttttaaaga | 2160 |
| acagattgga gcaggagtgg gatggagtct gaagtgtggg gctcagcctt ggggaggctt | 2220 |
| cgtaaagtcc agggagaaga caaagtcctg gtgactgtgg gtctaagcct gatactgact | 2280 |
| acttccctgg gcttctttct caacagataa gtgcattcaa gagtaaacac aggtgagaga | 2340 |
| agtcagtcac aggtaacaca tggtaagtgc catttactca ctcaacataa gaccactgag | 2400 |
| tgctcatgtg accacggagt gcgggctggg gtggggggga tgcagctgcc caaggactgt | 2460 |
| ccaagtgaga cagccagaga gaaaggacag ttccaattcc agtggcagga atagagctga | 2520 |
| tggccaaggg ttcatgggag aaggataaca gcaatgggaa gggaccgccc catgaagccc | 2580 |
| atcctgcaaa atgagtctcc aaggaaccag aatggacaag atcgggaagg gactggtggc | 2640 |
| cagggatgga catggcgagt cagagggctg gctcctcacc tgtgctgttg actgagactc | 2700 |
| tgagaccata ggccctggag ggatacccta ggaggccctg gccggaagtg ttgtttgggc | 2760 |
| cccactgggc tcagggtgct gccctcatca ctgatggctc ttgttcttct gtgcaggtga | 2820 |
| tgtggcctca ggactcccgt gctctgtcac tcttgagacc caagccctgg ctccccaaag | 2880 |
| accttctccg ccctccagct ttgccttggt ggagaaataa aatccaaag | 2929 |
| END | |

What is claimed is:

1. A method for constructing a Ptgds gene knockout rat model with spontaneous kidney yin deficiency, comprising the following steps:
1) designing two target sequences Ptgds-sgRNA1 and Ptgds-sgRNA2 at a Ptgds gene locus;
2) obtaining purified Cas9mRNA, purified Ptgds-sgRNA1, and purified Ptgds-sgRNA2 by in vitro transcription;
3) conducting targeted knockout on a 2,944 bp sequence fragment in the Ptgds gene using a CRISPR/Cas9 system to obtain a Ptgds knockout gene;
4) injecting the purified Cas9mRNA, the purified Ptgds-sgRNA1, the purified Ptgds-sgRNA2, and the Ptgds knockout gene into rat embryos, and transplanting the embryos into fallopian tubes of surrogate recipient rats to obtain neonatal rats;
5) conducting genetic identification on the neonatal rats to select heterozygous rats; and
6) conducting breeding on the heterozygous rats with wild-type rats for multiple generations, and subjecting offspring rats obtained from each generation to gene identification until obtaining homozygous rats, the obtained homozygous rats are Ptgds gene knockout rat model.

2. The method according to claim 1, wherein in step 1), the Ptgds-sgRNA1 has a nucleotide sequence set forth in SEQ ID NO: 1; and the Ptgds-sgRNA2 has a nucleotide sequence set forth in SEQ ID NO: 3.

3. The method according to claim 2, wherein in step 3), the sequence fragment comprises an intron sequence fragment and an exon sequence fragment.

4. The method according to claim 3, wherein in step 3), the intron sequence fragment has a nucleotide sequence set forth in SEQ ID NO: 5; and the exon sequence fragment has a nucleotide sequence set forth in SEQ ID NO: 6.

5. The method according to claim 4, wherein the gene identification in step 5) and step 6) comprises the following steps:
S1) extracting a genomic DNA from the neonatal rat;
S2) conducting PCR amplification with specific primers using the genomic DNA as a template to obtain an amplification product;
S3) conducting electrophoresis detection on the amplification product using agarose gel; and
S4) identifying the heterozygous rats or the homozygous rats according to an electrophoresis result.

6. The method according to claim 5, wherein in step S2), the specific primers comprise primers of Ptgds-L-S, Ptgds-L-A, Ptgds- R-S, and Ptgds-R-A, with nucleotide sequences set forth in SEQ ID NO: 7, SEQ ID NO: 8, SEQ ID NO: 9, and SEQ ID NO: 10, respectively.

7. The method according to claim 6, wherein in step S2), a reaction system of the PCR amplification comprises: 2.5 μl of a template DNA at 500 ng/μl, 2.5 μl of each of the Ptgds-L-S, the Ptgds-L-A, the Ptgds-R-S, and the Ptgds-R-A that are at 10 μmol/L, 5 μl of a 10× buffer, 5 μl of dNTP at 2.5 mmol/L, 0.5 μl of Eazy-taq, and supplementing to 50 μl with water; and
the PCR amplification comprises: pre-denaturation at 98° C. for 2 min; denaturation at 98° C. for 20 sec×30; annealing at 55° C. for 20 sec×30; extension at 72° C. for 10 sec×30; terminal extension at 72° C. for 5 min; and cooling at 16° C. for 2 min.

8. The method according to claim 7, wherein the step 6) specifically comprises the following steps:
6.1) using the heterozygous rats selected in step 5) as F0-generation heterozygous rats, caging with the wild-type rats, conducting gene identification on obtained offspring I, and selecting heterozygous rats from the offspring I as F1-generation rats;
6.2) caging the F1-generation rats with the wild-type rats, conducting gene identification on obtained offspring II, and selecting heterozygous rats from the offspring II as F2-generation rats; and
6.3) using rats generated by conducting self-breeding within a group of F2-generation rats as F3-generation rats, and conducting gene identification to select homozygous rats in F3-generation as Ptgds gene knockout rat model.
